(19) United States
(12) Patent Application Publication
(10) Pub. No.: US 2025/0161123 A1
(43) Pub. Date: May 22, 2025

(54) ABSORBENT PRODUCTS HAVING IMPROVED PACKAGING EFFICIENCY

(71) Applicant: The Procter & Gamble Company, Cincinnati, OH (US)

(72) Inventors: Naka SEIDEL, Frankfurt (DE); Christopher Philip BEWICK-SONNTAG, Cincinnati, OH (US); Florian Phillip ROUSSELANGE, SCHOENBORNRING (DE); Rosa LEI, Idstein (DE); Alejandro NAVARRO, Idstein (DE)

(21) Appl. No.: 18/950,728

(22) Filed: Nov. 18, 2024

Related U.S. Application Data
(60) Provisional application No. 63/600,887, filed on Nov. 20, 2023.

(51) Int. Cl. A61F 13/472; A61F 13/15; A61F 13/53

(52) U.S. Cl. CPC A61F 13/472; A61F 13/53; A61F 2013/15325; A61F 2013/530481

(57) ABSTRACT

A feminine hygiene product comprising a package having an interior space and an exterior surface and a plurality of disposable feminine hygiene pads disposed within the interior space of the package. Each of the disposable feminine hygiene pads having an absorbent core structure comprising an upper nonwoven layer, a lower nonwoven layer, and an inner core layer comprising cellulosic fibers. At least a portion of the inner core layer is disposed between the upper nonwoven layer and the lower nonwoven layer. The disposable feminine hygiene pads exhibit an Average In-Bag Pad Density of about 0.20 g/cm³ or less, and a % Caliper Recovery at 2 minutes of at least 4%, as measured according to the In Bag Compression Recovery Method.

### CROSS REFERENCE TO RELATED APPLICATION

This application claims the benefit of U.S. Provisional Application No. 63/600,887, filed Nov. 20, 2023, which is incorporated by reference herein in its entirety.

### FIELD OF THE INVENTION

The present disclosure relates generally to absorbent products, and more particularly, to packages containing flexible, conformable disposable absorbent articles that exhibit improved packaging efficiency, resulting in smaller, more environmentally friendly products.

### BACKGROUND OF THE INVENTION

Absorbent articles are widely used among consumers, e.g., diapers, training pants, feminine pads, adult incontinence pads, etc. Generally, absorbent articles such as these comprise a topsheet and a backsheet, with an absorbent core structure disposed therebetween. These absorbent articles are designed to absorb and retain liquids and other discharges from the human body to prevent body and garment soiling.

Historically, absorbent core structures for feminine pad applications utilize cellulose fibers in various ways to manage the complex and varied rheological properties of menstrual fluid and vaginal discharge. Traditional cellulose based absorbent core structures are thick and can be stiff, bulky and uncomfortable to wear. Over time, these thick cellulose rich absorbent cores have been made thinner with the inclusion of absorbent polymer materials, such as absorbent gelling material (“AGM”), to further boost their absorption properties. However, these absorbent core structures are less mechanically strong and even less able to retain their shape, particularly when loaded with liquid exudate. These thinner structures also tend to be densified (thus stiffer) and are often wrapped in a simple cellulose tissue or thin nonwoven layer to keep the AGM inside the core structure. Other approaches combine these wrapped cellulose and AGM cores with an additional fluid acquisition-distribution layer. However, these acquisition-distribution layers are not ideal for complex viscous fluids which need to move over the boundary between these layers. To better facilitate fluid partitioning from the acquisition-distribution system to the core, the core is often densified to increase capillarity and increase the ability of the core to pull fluid effectively from the acquisition-distribution layer above. Such densification of the absorbent system comes at the cost of comfort (stiffness) and the ability of the absorbent core structure and/or absorbent article to readily conform to the wearer's unique anatomical geometry.

While a relatively low density, flexible, and conformable cellulose based absorbent core structure is desirable for comfort and performance, it is difficult to sustain the intended density of such cores during manufacturing, packaging, shipping, and storage. It is well known that cellulose rich absorbent core structures are sensitive to compression during manufacture and/or packaging processes. For instance, holding a typical cellulose rich absorbent core structure in a compressed state over an extended period of time, such as in a package, can cause the article and/or the absorbent core structure to lose the ability to recover to its pre-compressed state. This is a process referred to as material (or product) creep as the system re-adjusts to extended periods of mechanical compression or stress to find a new equilibrium state. Absorbent articles that are held in a package under compression can become more densified, resulting in a stiff article that is less able to conform to the user's anatomical features and can be uncomfortable to wear. Additionally, extended compression can reduce absorbency efficiency, resulting in an article that feels wet and can exhibit larger stain sizes. Absorbent articles are also typically compressed during packaging in order to decrease the volume to store, ship, and reduce the space needed on store shelves. While product compression may increase packaging efficiency, it typically does so at the expense of product flexibility, conformability, and/or absorbency.

There is a need for absorbent articles that can provide a comfortable, conformable fit that can be compressed during manufacturing and packaging, yet can still substantially recover to their pre-compressed state. There further exists a need for a mechanism to fully optimize product delivery and shipping for such articles (optimization from the point of view of more articles per unit volume and less packaging per number of articles packed). These types of shipping efficiencies can reduce the environmental impact of shipping such articles by reducing the number of pallets and the number of trucks needed to ship the articles to various store locations and warehouses.

### SUMMARY OF THE INVENTION

The present disclosure reduces the cost and environmental impact of packing and shipping cellulose based absorbent articles with conforming features. The absorbent articles of the present disclosure comprise absorbent core structures comprising upper and lower nonwoven layers that sandwich an inner core layer comprising a liquid absorbent material comprising cellulosic fibers. Absorbent articles described herein can be compressed and held in a package yet maintain a relatively low density and substantially recover their pre-compressed caliper after removal from the package.

Disclosed herein is an absorbent product comprising: a package comprising an interior space and an exterior surface. A plurality of disposable feminine hygiene pads are disposed within the interior space of the package. Each of the disposable feminine hygiene pads comprising a topsheet, a backsheet, and an absorbent core structure disposed between the topsheet and the backsheet, wherein the absorbent core structure comprises an upper nonwoven layer, a lower nonwoven layer, and an inner core layer comprising cellulosic fibers, wherein at least a portion of the inner core layer is disposed between the upper nonwoven layer and the lower nonwoven layer. The disposable feminine hygiene pads exhibit an Average In-Bag Pad Density of about 0.20 g/cm³ or less, and a % Caliper Recovery at 2 minutes of at least 4%, as measured according to the In Bag Compression Recovery Method.

Also disclosed herein is an absorbent product comprising: a package comprising an interior space and an exterior surface. A plurality of disposable feminine hygiene pads are disposed within the interior space of the package. Each of the disposable feminine hygiene pads comprising a topsheet, a backsheet, and an absorbent core structure disposed between the topsheet and the backsheet, the absorbent core structure comprising an upper nonwoven layer comprising polymer fibers and having a basis weight of from about 30 gsm to about 85 gsm, a lower nonwoven layer comprising polymer fibers and having a basis weight of from about 7 gsm to about 40 gsm, and an inner core layer comprising a mixture of cellulosic fibers and superabsorbent particles, wherein at least a portion of the inner core layer is disposed between the upper nonwoven layer and the lower nonwoven layer. The feminine hygiene pads exhibit an Average In-Bag Pad Density of 0.20 g/cm³ or less, measured according to the In Bag Compression Recovery Method.

Also disclosed herein is an absorbent product comprising: a package comprising an interior space and an exterior surface. A plurality of disposable feminine hygiene pads are disposed within the interior space of the package. Each of the disposable feminine hygiene pads comprising a topsheet, a backsheet, and an absorbent core structure disposed between the topsheet and the backsheet, the absorbent core structure comprising an upper nonwoven layer comprising polymer fibers and having a basis weight of from about 30 gsm to about 85 gsm, a lower nonwoven layer comprising polymer fibers, and an inner core layer comprising from about 50% to about 85% cellulosic fibers, by weight of the inner core layer, and from about 15% to about 50% superabsorbent particle, by weight of the inner core layer, wherein at least a portion of the inner core layer is disposed between the upper nonwoven layer and the lower nonwoven layer. The feminine hygiene pads exhibit a % Caliper Recovery at 2 minutes of from about 4% to about 35%, as measured according to the In Bag Compression Recovery Method.

Also disclosed herein is a process for packaging a plurality of disposable feminine hygiene pads, the process comprising: providing a plurality of disposable feminine hygiene pads, each of the disposable feminine hygiene pads comprising a topsheet, a backsheet, and an absorbent core structure disposed between the topsheet and the backsheet, the absorbent core structure comprising an upper nonwoven layer comprising polymer fibers and having a basis weight of from about 30 gsm to about 85 gsm, a lower nonwoven layer comprising polymer fibers and having a basis weight of from about 7 gsm to about 40 gsm, and an inner core layer comprising cellulosic fibers and superabsorbent particles, wherein at least a portion of the inner core layer is disposed between the upper nonwoven layer and the lower nonwoven layer; folding each disposable feminine hygiene pad of the plurality of disposable feminine hygiene pads to form a plurality of folded disposable feminine hygiene pads; arranging the plurality of folded disposable feminine hygiene pads to form a stack of folded disposable feminine hygiene pads; compressing the stack of folded disposable feminine hygiene pads along a compression axis to form a compressed stack of folded disposable feminine hygiene pads; placing the compressed stack of folded disposable feminine hygiene pads in an interior space of a package, wherein the compressed stack of folded disposable feminine hygiene pads is placed in the interior space of the package such that the compression axis of the stack of folded disposable feminine hygiene pads is oriented substantially along the width dimension of the package; and closing the package such that the folded disposable feminine hygiene pads exhibit an Average In-Bag Folded Caliper from about 7.0 to about 15.0 mm, and such that upon removal from the package, the disposable feminine hygiene pads exhibit a % Caliper Recovery at 2 minutes of at least 4%, as measured according to the In Bag Compression Recovery Method.

### BRIEF DESCRIPTION OF THE DRAWINGS

FIG. 1 is a representation of an absorbent core structure in accordance with the present disclosure.

FIG. 2 is a cross-sectional view of the absorbent core structure taken along line 2-2 of FIG. 1.

FIG. 3 is a plan view of an absorbent article, wearer-facing surface facing the viewer, with a portion of the structure being cut-away to more clearly show the construction of the absorbent core structure.

FIG. 4 is a cross-sectional view taken along lines 4-4 of FIG. 3.

FIG. 5 is a plan view of an absorbent article, wearer-facing surface facing the viewer, illustrating example absorbent core structure arrangements.

FIG. 6A is a cross-sectional view of the absorbent article taken along line 6A-6A of FIG. 5.

FIG. 6B is a cross-sectional view of the absorbent article taken along line 6B-6B of FIG. 5.

FIG. 7 is a cross-sectional view of the absorbent article taken along line 7-7 of FIG. 5.

FIG. 8 is a plan view of an absorbent article, wearer-facing surface facing the viewer, with the topsheet removed illustrating example inner core layer and adhesive arrangements.

FIG. 9 is a close up illustration of a structural bond site, according to one or more configurations shown and described herein.

FIG. 10 is a cross-sectional view of the structural bond site of FIG. 9.

FIG. 11A is a plan view of an example absorbent article, wearer-facing surface facing the viewer, illustrating flex bond channel regions, according to one or more configurations shown and described herein.

FIG. 11B is a plan view of an example absorbent article, wearer-facing surface facing the viewer, illustrating flex bond channel regions, according to one or more configurations shown and described herein.

FIG. 12A is side view of a package of absorbent articles in accordance with the present disclosure showing the filled bag width of the package. The outer surface is illustrated as transparent for purposes of clarity.

FIG. 12B is a side view of a package of absorbent articles in accordance the present disclosure showing the filled bag height of the package. The outer surface is illustrated as transparent for purposes of clarity.

FIG. 13 is a perspective view of a package of absorbent articles in accordance the present disclosure showing the filled bag depth of the package.

FIGS. 14A-C are a test method arrangement for the Wet and Dry CD Ultra Sensitive 3 Point Bending Method.

FIGS. 15, 16A, and 16B are the test method arrangement for the Wet and Dry Bunched Compression Test.

FIGS. 17A and 17B are illustrative graphs of Bunch Curves resulting from the Wet and Dry Bunched Compression Test. The graphs in FIGS. 17A and 17B are shown to illustrate how the calculations in the method may be performed and do not represent the data described herein.

FIG. 18 is a top view of a strikethrough plate used in the Acquisition Time and Rewet Method described herein.

FIG. 19 is a bottom view of the strikethrough plate used in the Acquisition Time and Rewet Method described herein.

FIG. 20A is a cross section view of the strikethrough plate used in the Acquisition Time and Rewet Method described herein, taken along a plane defined by the z-direction and line 20A-20A shown in FIG. 18.

FIG. 20B is a cross section view of the strikethrough plate used in the Acquisition Time and Rewet Method described herein, taken along a plane defined by the z-direction and line 20B-20B shown in FIG. 18.

### DETAILED DESCRIPTION OF THE INVENTION

As used herein “disposable absorbent article” or “absorbent article” shall be used in reference to articles such as diapers, training pants, diaper pants, refastenable pants, adult incontinence pads, adult incontinence pants, feminine hygiene pads, cleaning pads, and the like, each of which are intended to be discarded after use.

As used herein “absorbent core structure” shall be used in reference to the upper nonwoven layer, the lower nonwoven layer, and the inner core layer disposed between the upper nonwoven layer and the lower nonwoven layer. As used herein, “absorbent core structure” does not include any secondary topsheet, topsheet, secondary backsheet, or backsheet of the absorbent article.

As used herein “hydrophilic” and “hydrophobic” have meanings as well established in the art with respect to the contact angle of water on the surface of a material. Thus, a material having a water contact angle of greater than about 90 degrees is considered hydrophobic, and a material having a water contact angle of less than about 90 degrees is considered hydrophilic. Compositions which are hydrophobic, will increase the contact angle of water on the surface of a material while compositions which are hydrophilic will decrease the contact angle of water on the surface of a material. Notwithstanding the foregoing, reference to relative hydrophobicity or hydrophilicity between a material and a composition, between two materials, and/or between two compositions, does not imply that the materials or compositions are hydrophobic or hydrophilic. For example, a composition may be more hydrophobic than a material. In such a case neither the composition nor the material may be hydrophobic; however, the contact angle exhibited by the composition is greater than that of the material. As another example, a composition may be more hydrophilic than a material. In such a case, neither the composition nor the material may be hydrophilic; however, the contact angle exhibited by the composition may be less than that exhibited by the material.

As used herein, the term “filament” refers to any type of continuous strand produced through a spinning process, a meltblowing process, a melt fibrillation or film fibrillation process, or an electrospinning production process, or any other suitable process to make filaments. The term “continuous” within the context of filaments are distinguishable from staple length fibers in that staple length fibers are cut to a specific target length. In contrast, “continuous filaments” are not cut to a predetermined length, instead, they can break at random lengths but are usually much longer than staple length fibers.

As used herein, “machine direction” refers to the direction in which a web flows through an absorbent article converting process. For the sake of brevity, may be referred to as “MD”.

As used herein, “cross machine direction” refers to the direction which is perpendicular to the MD. For the sake of brevity, may be referred to as “CD”.

As used herein, “resilient” refers to a material that tends to retain its shape both in the dry and wet states and when subjected to a compression force tends to recover its original, pre-compression shape when such force is removed. In some aspects, the upper and/or lower nonwoven layers described herein may be resilient.

“Decitex” also known as Dtex is a measurement used in the textile industry used for measuring yarns or filaments. 1 Decitex=1 gram per 10,000 meters. In other words, if 10,000 linear meters of a yarn or filament weights 500 grams that yarn or filament would have a decitex of 500.

As used herein, “wearer-facing” (sometimes referred to herein as body-facing) and “outward-facing” (sometimes referred to herein as garment-facing) refer respectively to the relative location of an element or a surface of an element or group of elements. “Wearer-facing” implies the element or surface is nearer to the wearer during wear than some other element or surface. “Outward-facing” implies the element or surface is more remote from the wearer during wear than some other element or surface (i.e., element or surface is proximate to the wearer's garments that may be worn over the absorbent article).

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The present disclosure relates to disposable absorbent articles comprising an absorbent core structure which comprises an upper nonwoven layer and a lower nonwoven layer, with an inner core layer comprising a liquid-absorbent material disposed between the upper and lower nonwoven layers. The liquid-absorbent material may comprise a homogeneous mixture of cellulosic fiber and superabsorbent particles, sometimes referred to herein as “fluff/AGM”. At least a portion of the inner core layer may be contained within the upper and lower nonwoven layers by sealing a portion of the first and second side regions of the upper nonwoven layer with the first and second side regions of the lower nonwoven layer to define a perimeter seal where adhesive is positioned between the upper nonwoven layer and the lower nonwoven layer and bonds the layers together. In some configurations, the perimeter seal may extend around the entire perimeter of the inner core layer.

The absorbent core structure described herein is configured to compress and recover its original shape (dry or wet) across a range of bodily movements and compressions. The flexibility and/or resiliency of the absorbent core structure results in an absorbent article that comfortably conforms to the wearer's anatomical geometry while efficiently managing the fluid as it exits the body. This can, unexpectedly, be achieved without typical densification stiffening (for wet integrity) by leveraging resilient upper and lower nonwoven layers composed of resilient polymers located above and below the loosely packed liquid-absorbent material of the inner core layer. The absorbent core structure is surprisingly able to carry the structural load and recover shape without physically being stiff or losing the desired structural properties when the absorbent core structure becomes wet. Without being limited by theory, it is believed that wet integrity/shape stability in a cellulose rich absorbent core structure without substantial densification and stiffening may be achieved when select resilient upper and lower nonwovens are positioned above and below the liquid-absorbent material of the inner core layer and are joined to and around the liquid-absorbent material. The upper and lower nonwovens may have sufficient recovery force to carry the liquid-absorbent material back to the original state and/or a stable fiber orientation state following compression. Wrapping or encapsulating a cellulose rich pulp core with a simple cellulose tissue or less resilient nonwoven material may not exhibit sufficient recovery energy to recover shape in-use and particularly when wetted. Structural, wet resilient nonwovens detailed herein may exhibit recovery energies following compression that are sufficient to recover the cellulose rich fiber matrix and are chosen to deliver high compression recovery, with relatively low stiffness, in both dry and wet states. It is believed that suitable absorbent core structures have a low force to compress (less resistance) and the structure is able to substantially recover its shape as the user, in a cyclic fashion, compresses and releases the compressive force with various body movements. To achieve this, the structure should sustain sufficient recovery energy following multiple cyclic compressions. Without sufficient recovery energy, the structure will remain in a compressed bunched state with insufficient force (stored energy) to recover.

Absorbent articles are typically aggressively compressed during manufacturing and packaging to help minimize shipping and/or warehousing costs. Typical fluff and airlaid absorbent products are manufactured to have a densified absorbent system, so compression during manufacturing and/or in packaging is not typically a concern for manufacturers because it does not significantly impact the intended product caliper, density, and/or performance. However, in the case of a relatively low density, flexible absorbent article and/or absorbent core structure such as described herein, aggressive compression may significantly impact the ability of the absorbent article and/or absorbent structure to recover caliper after compression has been removed and may create a wet feel and an increase in stain size during use. It was surprisingly found that the absorbent article and/or absorbent core structure described herein can be compressed at relatively high levels and held in a package without permanent densification and the absorbent article and/or absorbent core structure can substantially recover its pre-compressed caliper after removal from the package. Without being limited by theory, it is believed that the increase in compressibility provides multiple cost-savings benefits, such as lower shipping costs, lower storage/warehousing costs, reduced packaging costs, reduced shelving/stocking costs, etc., and provides smaller package sizes resulting in more environmentally friendly packaging.

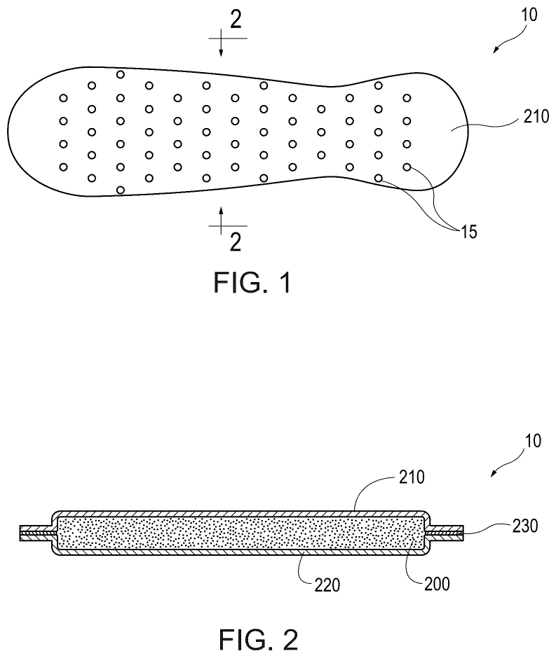

An exemplary absorbent core structure 10 of the present disclosure is represented in FIG. 1. FIG. 2 is a cross-sectional view taken along lines 2-2 of FIG. 1 with the structural bond sites 15 removed to more clearly show the absorbent core structure 10.

Referring to FIGS. 1 and 2, absorbent core structure 10 may comprise an upper nonwoven layer 210 and a lower nonwoven layer 220 (also referred to herein collectively as upper and lower nonwoven layers or upper and lower nonwovens) and an inner core layer 200 disposed between the upper nonwoven layer 210 and the lower nonwoven layer 220. The absorbent core structure 10 may comprise inner core layer 200 comprising a liquid-absorbent material. The liquid absorbent material may comprise a homogeneous mixture of cellulose pulp and superabsorbent particles. A portion of the upper and lower nonwoven layers 210, 220 may be joined together at a perimeter seal 230.

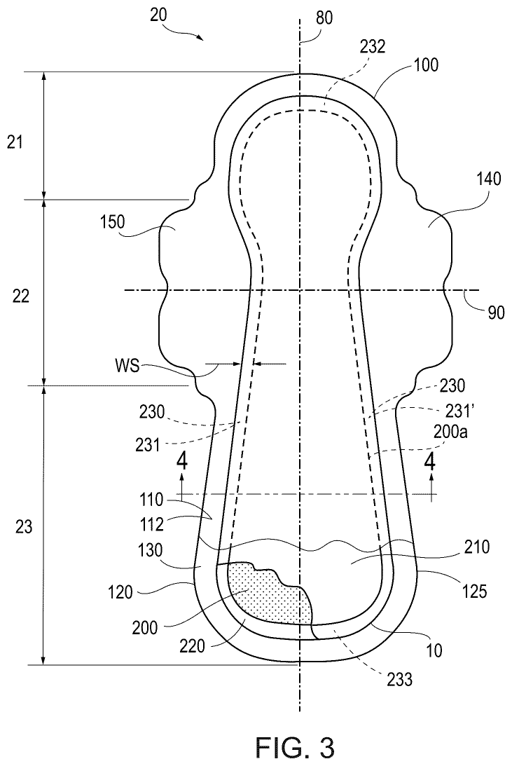

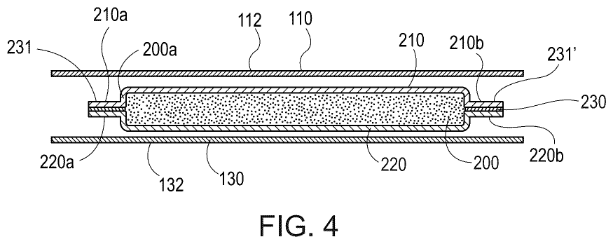

An exemplary absorbent article 20 in the form of a feminine hygiene pad is represented in FIG. 3. To provide a frame of reference for the present discussion, the absorbent article 20 of FIG. 3 is shown with a longitudinal axis 80, a lateral axis 90. FIG. 3 is a plan view of an absorbent article 20, wearer-facing surface 112 facing the viewer, with a portion of the structure being cut-away to more clearly show the construction of the absorbent core structure 10. FIG. 4 is a cross-sectional view of absorbent article 20 taken along line 4-4 of FIG. 3.

Referring to FIGS. 3-4, the absorbent article 20 may comprise a topsheet 110, a backsheet 130, and the absorbent core structure 10 disposed between the topsheet 110 and the backsheet 130. Absorbent article 20 may comprise a wearer-facing surface 112 and a garment-facing surface 132. Absorbent article 20 and absorbent core structure 10 may each include a front region 21, a rear region 23, and a middle region 22 disposed intermediate the front region 21 and the rear region 23. In some configurations, the absorbent core structure 10 may have a non-rectangular perimeter. In particular, the absorbent core may be shaped to define a tapering along its width towards the middle region of the absorbent core structure. The absorbent core structure may conform to a wearer's inner thigh geometry, such as, for example, an hourglass shape, an offset hourglass shape (one end is wider than an opposite end and a narrowed mid-section between the ends), a bicycle seat shape (one end and central portion are narrower than the second end), an oval, or a trapezoid shape.

In some configurations, the disposable absorbent article 20 may comprise the following structure (from a wearer-facing surface to an outward-facing surface): a topsheet 110, an upper nonwoven layer 210, an inner core layer 200, a lower nonwoven layer 220, and a backsheet 130. In some aspects, the topsheet 110 may be in direct contact with the upper nonwoven layer 210, the upper nonwoven layer 210 may be in direct contact with the inner core layer 200, and/or the inner core layer 200 may be in direct contact with the lower nonwoven layer 220. By “direct contact”, it is meant that there is no further intermediate component layer between the respective layer in direct contact thereto. It is however not excluded that an adhesive material may be disposed between at least a portion of the layers described above.

Upper nonwoven layer 210 may comprise a first side region 210a and a laterally opposing second side region 210b, and lower nonwoven layer 220 may comprise a first side region 220a and an opposing second side region 220b. In some configuration, the first side regions 210a, 220a of the upper nonwoven layer and the lower nonwoven layer may extend substantially parallel to the longitudinal axis 80. The upper and lower nonwoven layers 210, 220 may extend outwardly from an inner core layer perimeter 200a and may be joined together with glue or other conventional bonding methods including, but not limited to, ultrasonic bonding, fusion bonding, crimping, and combinations thereof, to form perimeter seal 230. In some configurations, the entire inner core layer 200 may be located inboard of the perimeter seal 230. The perimeter seal 230 may help to seal the liquid-absorbent material of the inner core layer 200 inside the upper and lower nonwoven layers 210, 220. Perimeter seal 230 may comprise at least a first seal region 231 extending substantially parallel to the longitudinal centerline 80 and a second seal region 231′ opposite the first seal region 231. In some configurations, perimeter seal 230 may further comprise a front perimeter seal region 232 and/or a back perimeter seal region 233. In some configurations, the perimeter seal 230 may extend around the entire inner core layer perimeter 200a.

Without being limited by theory, it is believed that resilient nonwoven layers comprising polymer fibers may hold their shape and resist plasticizing when wet when attached to the inner core layer through the application of a core construction adhesive that is applied either directly to the inner core layer or the resilient nonwoven layer(s) via a conventional spray coating application chosen to achieve a bond but not disrupt the flow of fluid to the inner core layer. The perimeter seal 230 may be positioned in at least the middle region 22 of the absorbent article 20 and/or the absorbent core structure 10. It is believed that the middle region 22 (located between the wearer's thighs during use) may be subjected to the most frequent and/or highest forces during use. It was found that the presence of at least a partial perimeter seal at a first side region and a second side region of the upper nonwoven layer and the lower nonwoven layer external to the inner core layer may help to ensure the upper and lower nonwovens maintain their structural function during physical deformations without separating, limiting any potential integrity and bunching issues. In addition, a perimeter seal may allow for any excess nonwoven material to be removed in order to enable an absorbent core structure to be shaped to conform to inner thigh geometry.

The perimeter seal 230 may have a seal width WS of between about 1 mm and about 10 mm, or between about 2 mm and about 8 mm, or between about 3 mm and about 6 mm. The seal width WS may be uniform or may vary about the perimeter of the inner core layer.

In some configurations, the upper and lower nonwoven layers 210, 220 may be discrete materials that can be cut to approximately the size and shape of the inner core layer 200 so as to fit between the topsheet 110 and backsheet 130. In some configurations, the inner core layer 200, upper nonwoven layer 210 and/or lower nonwoven layer 220 may be shaped, meaning it is non-rectangular. In some configurations, the upper and/or lower nonwoven layers 210, 220 may be rectangular.

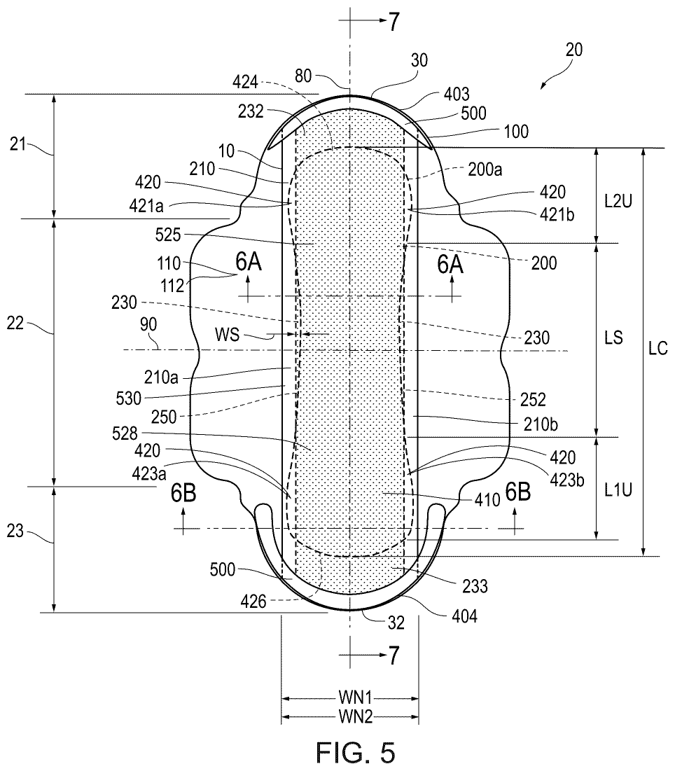

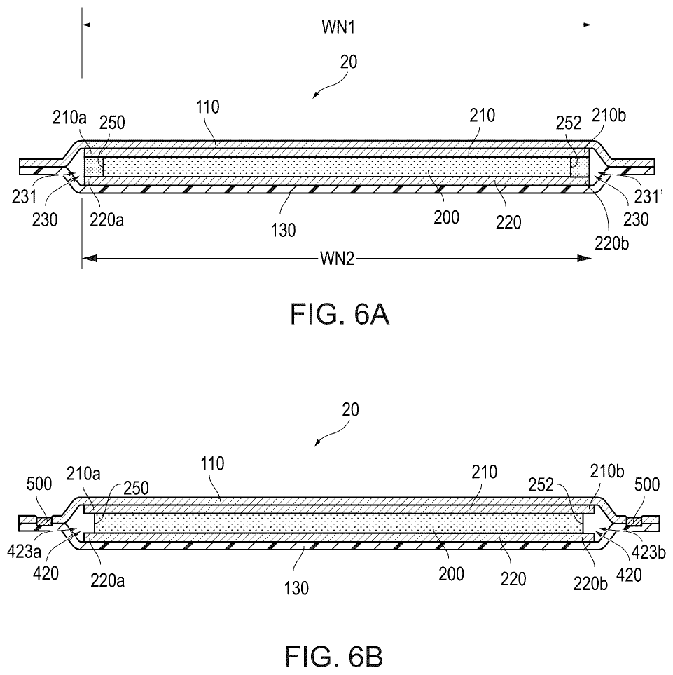

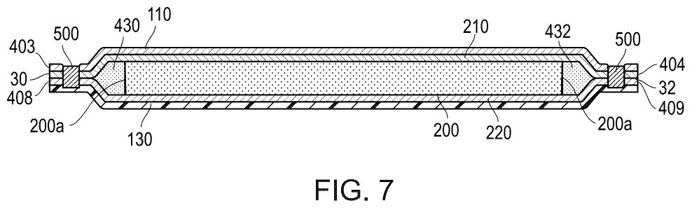

In some configurations, portions of the absorbent core structure may not comprise a perimeter seal. In some configurations, the perimeter seal 230 may extend only partially around the inner core layer perimeter 200a. FIGS. 5-8 show an absorbent article illustrating example absorbent core structure arrangements. Elements of FIGS. 5-8 having the same reference number as described above with respect to FIGS. 1-4 may be the same element (e.g., inner core layer 200). FIG. 6A is a cross-sectional view of the absorbent article taken along line 6A-6A of FIG. 5. FIG. 6B is a cross-sectional view of the absorbent article taken along line 6B-6B of FIG. 5. FIG. 7 is a cross-sectional view of the absorbent article taken along line 7-7 of FIG. 5.

Referring to FIGS. 5-7, in some configurations, the upper nonwoven layer 210 may extend longitudinally between a front edge 403 and a back edge 404 and define first side region 210a and laterally opposing second side region 210b. The lower nonwoven layer 220 may extend longitudinally between a front edge 408 and a back edge 409 and define first side region 220a and laterally opposing second side region 220b. The upper nonwoven layer 210 may have a first nonwoven lateral width WN1, and the lower nonwoven layer 220 may have a second nonwoven lateral width WN2. In some configurations, the first and second nonwoven lateral widths WN1, WN2 may be substantially the same. In some configurations, the first and second nonwoven lateral width WN1, WN2 may be different. The first nonwoven lateral width WN1 and/or the second nonwoven lateral width WN2 may be from about 40 mm to about 110 mm, or from 45 mm to about 90 mm, or from about 50 mm to about 80 mm. The upper and/or lower nonwoven layers 210, 220 may have a longitudinal length of from about 100 mm to about 450 mm, or from about 150 mm to about 375 mm. In some configurations, the upper and/or lower nonwoven layers 210, 220 may extend from a front article edge 30 to a back article edge 32.

At least a portion of the inner core layer 200 may be disposed between the upper nonwoven layer 210 and the lower nonwoven layer 210. In some configurations, the entire inner core layer 200 may be disposed between the upper nonwoven layer 210 and the lower nonwoven layer 220.

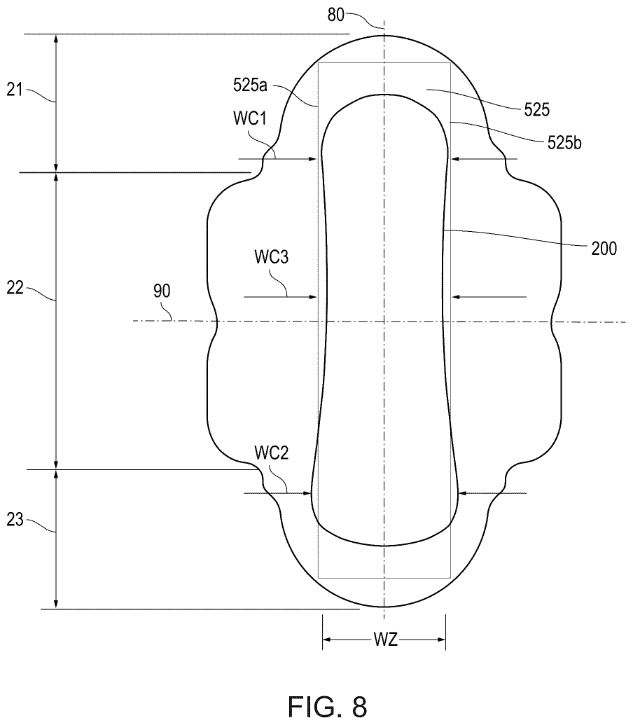

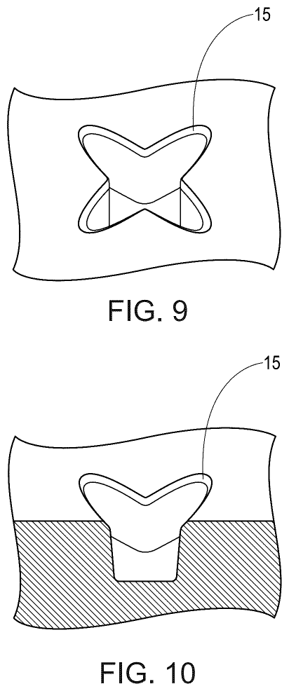

The inner core layer 200 extends longitudinally between an inner core layer front edge 424 and an inner core layer back edge 426 and extends laterally from a first side edge 250 and to a second side edge 252. In some configurations, the inner core layer 200 may be shaped. As shown in FIG. 8, the inner core layer 200 may define a first inner core layer lateral width, WC1, a second inner core layer lateral width, WC2, and a third inner core layer lateral width, WC3, disposed therebetween. In some configurations, the first inner core layer lateral width, WC1, may be in the front region 21 and the second inner core layer lateral width, WC2, may be positioned in the rear region 23. In some configurations, the third inner core layer lateral width, WC3, may be less than the first and second inner core layer lateral widths, WC1, WC2. In some configurations, the second inner core layer lateral width, WC2, may be greater than the first and third inner core layer lateral width, WC1, WC3. The first inner core layer lateral width, WC1, may be from about 50 to about 80 mm, the second inner core layer lateral width, WC2, may be from about 55 mm to about 100 mm, the third inner core layer lateral width, WC3, may be from about 40 mm to about 70 mm.

An adhesive zone 525 may be disposed intermediate at least one of the upper nonwoven layer 210 and the lower nonwoven layer 220 and the inner core layer 200. The adhesive zone 525 may comprise an adhesive 528 that extends from the first side region 210a of the upper nonwoven layer 210 to the second side region 210b of the upper nonwoven layer 210 and/or from the first side region 220a of the lower nonwoven layer 220 to the second side region 220b of the lower nonwoven layer 220. As shown in FIG. 8, the adhesive zone 525 may extend from a first edge 525a to a second edge 525b to define an adhesive zone lateral width, WZ, of from about 35 mm to about 110 mm, or from about 40 to about 105 mm. In some configurations, the first and second edge 525a, 525b of the adhesive zone 525 may be coterminous with or spaced laterally inboard from the lateral edges of the upper and/or lower nonwoven layers 210, 220. In some configurations, as shown in FIG. 5, gap regions 530 may be defined on the upper and/or lower nonwoven layers 210, 220 by the absence of adhesive 528 between the first and second edges 525a, 525b of the adhesive zone 525 and the edge of the nonwoven. The gap region 530 may have a width of less than 5 mm, or from about 0.1 mm to about 5 mm, or from about 0.5 to about 3 mm. In some configurations, the upper nonwoven layer 210 and the lower nonwoven layer 220 may substantially surround the adhesive zone 525 and the inner core layer 200.

In some configurations, a portion of the inner core layer 200 may be contained within the upper nonwoven layer 210 and the lower nonwoven layer 220 by sealing a portion of the first side region 210a and the second side region 210b of the upper nonwoven layer 210 with a portion of the first side region 220a and the second side region 220b of the lower nonwoven layer 220 to define a lateral perimeter seal 230 where adhesive 528 is positioned between the upper nonwoven layer 210 and the lower nonwoven layer 220. As such, adhesive 528 may bond the upper nonwoven layer 210 with the lower nonwoven layer 220. The lateral perimeter seal 230 may be positioned in the middle region 22 and may have a longitudinal seal length, LS, that is from about 45% to about 90% of a longitudinal inner core length, LC, or from about 50% to about 85%.

Referring to FIG. 5, a portion of the inner core layer 200 may extend laterally outboard of the adhesive zone 525 to define an unsealed portion 420. The unsealed portion 420 may be positioned longitudinally outboard of the perimeter seal 230. It is to be appreciated that the absorbent core structure 10 may comprise one or more unsealed portions 420, such as for example, two, three, or four unsealed portions, depending on the size and/or positioning of the upper and lower nonwoven layers in relation to the size and/or positioning of the adhesive zone and the inner core layer.

The absorbent core structure 10 may comprise a first unsealed portion 423a where a portion of the inner core layer 200 extends laterally outboard of the adhesive zone 525. In some configurations, the absorbent core structure 10 may comprise a second unsealed portion 423b where a second portion of the inner core layer 200 extends laterally outboard of the adhesive zone 525. Second unsealed portion 423b may be laterally separated from first unsealed portion 423a by a sealed portion 410. In some configurations, the first and second unsealed portions 423a, 423b may be positioned in the rear region 23 and may extend longitudinally into a portion of the middle region 22. The absorbent core structure 10 may further comprise a third unsealed portion 421a where a third portion of the inner core layer 200 extends laterally outboard of the adhesive zone 525. In some configurations, the absorbent core structure 10 may comprise a fourth unsealed portion 421b where a fourth portion of the inner core layer 200 extends laterally outboard of the adhesive zone 525. Fourth unsealed portion 421b may be laterally separated from third unsealed portion 421a by a sealed portion 410. In some configurations, the third and fourth unsealed portions 421a, 421b may be positioned in the front region 21 and may extend longitudinally into the middle region 22. It is to be understood that an unsealed portion 420 may also be formed in configurations wherein a portion of the inner core layer perimeter 200a is coterminous with a first or second edge 525a, 525b of the adhesive zone 525.

The first and second unsealed portions 423a, 423b may have an unsealed longitudinal length L1U that is about 5% to about 30%, or from about 8% to about 25%, of the longitudinal inner core length, LC. The third and fourth unsealed portions 421a, 421b may have an unsealed longitudinal length L2U that is about 5% to about 30%, or from about 8% to about 25%, of the longitudinal inner core length, LC. In some configurations, the unsealed longitudinal length L1U of the first or second unsealed portions 423a, 423b may be greater than the unsealed longitudinal length L2U of the third or fourth unsealed portions 421a, 421b.

FIGS. 6A and 6B are cross-sectional views of the absorbent article 20 of FIG. 5 taken along lines 6A-6A and 6B-6B, respectively, showing configurations of the absorbent core structure 10. In particular, FIG. 6A is a cross-sectional view through the middle region 22 of the absorbent article 20 showing upper and lower nonwoven layers 210, 220 extend laterally outboard of the first and second side edges 250, 252 of inner core layer 200. As discussed above, a portion of the inner core layer 200 may be contained within the upper nonwoven layer 210 and the lower nonwoven layer 220 by sealing a portion of the first side region 210a and the second side region 210b of the upper nonwoven layer 210 with a portion of the first side region 220a and the second side region 220b of the lower nonwoven layer 220 to define lateral perimeter seal 230. FIG. 6B is a cross-sectional view through the rear region 23 of the absorbent article 20 showing upper and lower nonwoven layers 210, 220 extend laterally outboard of the first and second side edges 250, 252 of inner core layer 200. As discussed above, a portion of the inner core layer 200 may extend laterally outboard of the adhesive zone 525 (not shown) to define unsealed portion 420. It is to be appreciated that the garment-facing surface of the upper nonwoven layer 210 and/or the wearer-facing surface of the lower nonwoven layer 220 may be coated with adhesive 528 to provide a connection with the inner core layer 200 and/or to form perimeter seal 230. Adhesive between the layers (except for in the perimeter seal) is not shown in FIGS. 6A and 6B for simplicity.

As previously mentioned, the upper and lower nonwoven layers 210, 220 may be further joined at a front perimeter seal region 232 and/or a back perimeter seal region 233 positioned longitudinally outboard of the inner core layer 200. The front perimeter seal region 232 and/or the back perimeter seal region 233 may extend longitudinally from an inner core layer perimeter 200a a distance of from about 3 mm to about 30 mm, or from about 5 mm to about 15 mm. Without being limited by theory, it is believed that front and/or back perimeter seal regions 232, 233 of less than about 3 mm may not provide a sufficient distance on the manufacturing line to avoid contamination of liquid absorbent material outside of the inner core layer. In some configurations, the front perimeter seal region 232 may be coterminous with or spaced longitudinally inboard from the front edge 403 of the upper nonwoven layer 210 and/or the front edge 406 of the lower nonwoven layer 220. In some configurations, the back perimeter seal 233 may be coterminous with or spaced longitudinally inboard from the back edge 404 of the upper nonwoven layer 210 and/or the back edge 409 of the lower nonwoven layer 220.

In some configurations, the first inner core layer lateral width WC1 and the adhesive zone width WZ may be substantially the same, creating an unsealed portion where adhesive 528 does not extend laterally outboard of the inner core layer perimeter 200a and the upper nonwoven layer 210 is not joined to the lower nonwoven layer 220 in this region. It is to be understood that in some configurations the second inner core layer lateral width WC2 may be substantially the same as the adhesive zone width WZ, thus defining an unsealed portion in the rear region 23. In some configurations, the first inner core layer lateral width WC1 may be less than the adhesive zone width WZ. Upper and lower nonwoven layers 210, 220 and adhesive zone 525 may substantially surround the inner core layer in the front region, and lateral perimeter seal 230 may extend longitudinally from the middle region 22 into the front region 21. In this configuration, the inner core layer is sealed within the upper and lower nonwoven layers 210, 220 in the middle region 22 and the front region 21. It is to be understood that in some configurations the second inner core layer lateral width WC2 may be less than the adhesive zone width WZ, thus creating a lateral perimeter seal 230 that extends longitudinally from the middle region 22 to the rear region 23.

In some configurations, at least one of the upper nonwoven layer 210 and the lower nonwoven layer 220 may be narrower than at least a portion of the inner core layer 210. In some configurations, the upper nonwoven layer 210 and/or lower nonwoven layer 220 may be narrower than the first inner core layer lateral width, WC1, and/or the second inner core layer lateral width, WC2.

Referring to FIGS. 5 and 7, the front edge 403 of the upper nonwoven layer 210 and/or the front edge 408 of the lower nonwoven layer 220 may be coterminous with or spaced longitudinally inboard from a front article edge 30. In some configurations, the back edge 404 of the upper nonwoven layer 210 and/or the back edge 409 of the lower nonwoven layer 220 may be coterminous with or spaced longitudinally inboard from a back article edge 32. The absorbent article 20 may further comprise crimp seal 500 positioned in the front region 21 and/or the rear region 23. In some configurations, the crimp seal 500 may extend from the front region 21 and/or the rear region 23 into the middle region 22. In some configurations, crimp seal 500 may be positioned longitudinally outboard of the front and back perimeter seals 232, 233. In some configurations, front and/or back perimeter seal regions 232, 233 may extend into crimp seal 500.

Crimp seal 500 may join the topsheet 110, the backsheet 130, and at least one of the upper nonwoven layer 210 and the lower nonwoven layer 220. In some configurations, crimp seal 500 may join topsheet 110 to backsheet 130. It was surprisingly found that crimp seal 500 may include upper and/or lower nonwoven layers 210, 220 without becoming stiff and uncomfortable. The crimp seal 500 may be substantially free of liquid absorbent material.

Referring to FIG. 3, the absorbent article 20 may further comprise a chassis 100 comprising the absorbent core structure 10. The absorbent core structure 10 and/or the inner core layer 200 may be shaped. Side edges 120 and 125 of the absorbent article 20 may follow the general contour of the absorbent core structure 10 and/or the inner core layer 200. So, for example, where the absorbent core structure 10 has an hourglass shape, the side edges 120, 125 of the absorbent article 20 may be arranged in an hourglass shape as well. However, forms are contemplated where the side edges 120 and 125 are generally straight or slightly curved such that they do not follow the contour of the absorbent core structure. In some configurations, the absorbent article 20 may be symmetric about the longitudinal centerline 80 or asymmetric about the longitudinal centerline 80. Similarly, the absorbent article 20 may be symmetric about the lateral centerline 90 or asymmetric about the lateral centerline 90.

In some configurations, as shown in FIGS. 1, 9, and 10, the absorbent article and/or the absorbent core structure may comprise a plurality of structural bond sites 15. FIGS. 9 and 10 show illustrations of example structural bond sites 15. FIG. 9 is a close up illustration of an example structural bond site 15. FIG. 10 is a cross-section view of the structural bond site 15 of FIG. 9.

The structural bond sites 15 may be symmetric and/or asymmetrical and may be any shape including, but not limited to, circles, ovals, hearts, diamonds, triangles, squares, stars, and/or X shapes. While the shape of the structural bond sites 15 may be any shape, suitable shapes may be more detailed shapes such as asymmetrical shapes (versus simple dots). The structural bond sites 15 may be on the absorbent article and/or on the absorbent core structure. In some configurations, the structural bond sites may have a bond area of from about 2 mm² to about 5 mm². In some configurations, the total structural bond area may be from about 0.5% to about 7.5%, or from about 0.75% to about 5%, or from about 1% to about 4% of the absorbent core structure, as measured according to the Structural Bond Sites Pattern Spacing and Area Measurement Method. In some configurations, the total structural bond area may be from about 1% to about 4% of absorbent article as measured according to the Structural Bond Sites Pattern Spacing and Area Measurement Method. The average distance between the structural bond sites may be from about 10 mm to about 32 mm. In some configurations, the average distance between the structural bond sites may be greater than about 20 mm. In some configurations, the structural bond sites may have a maximum width of from about 1 mm to about 6 mm, or from about 1.5 mm to about 5 mm, or from about 2 mm to about 4 mm. Without being limited by theory, it is believed that the average distance between structural bond sites and/or the size of the structural bond sites may help to maintain the structural integrity of the absorbent core structure without creating an undesirable stiffness that may inhibit the ability of the absorbent article to conform to the body.

In some configurations, the structural bond sites may be distributed across the absorbent article and/or absorbent core structure or they may be clustered in regions of the absorbent article and/or absorbent core structure. In some configurations, the structural bond sites may be clustered in the middle region 22 of the absorbent article and/or absorbent core structure. In some configurations, the middle region 22 of the absorbent article and/or absorbent core structure may be substantially free from structural bond sites and may be surrounded by an area of structural bond sites and/or embossing.

In some configurations, the structural bond sites 15 may join the topsheet 110, the upper nonwoven layer 210, the absorbent core structure 10, and the lower nonwoven layer 220. In some configurations, the structural bond sites 15 may join the upper nonwoven layer 210, the absorbent core structure 10, and the lower nonwoven layer 220. Absorbent articles 20 and/or absorbent core structures 10 may comprise an upper nonwoven layer 210 and a lower nonwoven layer 220 that are closer together in the Z-direction at the structural bond sites 15 but are not melted together. Since these structural bond sites are not melted together, they may not be permanent in nature and rather may intermingle the materials within the structural bond site. In some configurations, the structural bond sites 15 may be substantially free of fusion bonds.

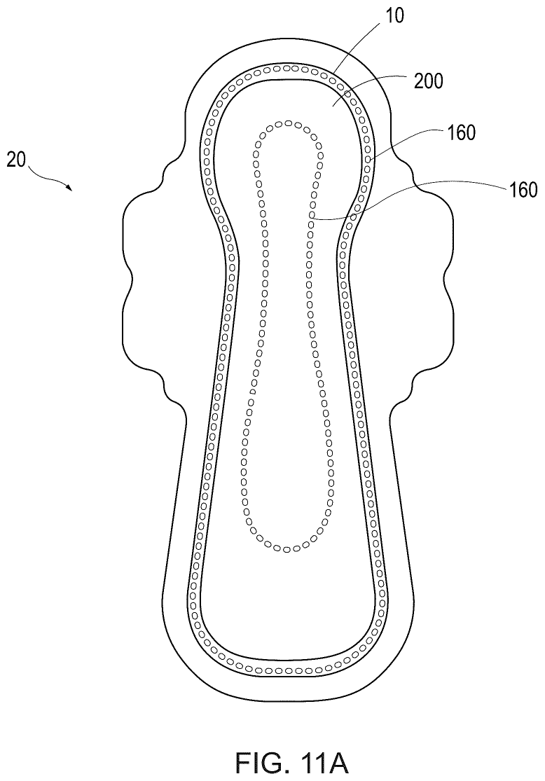

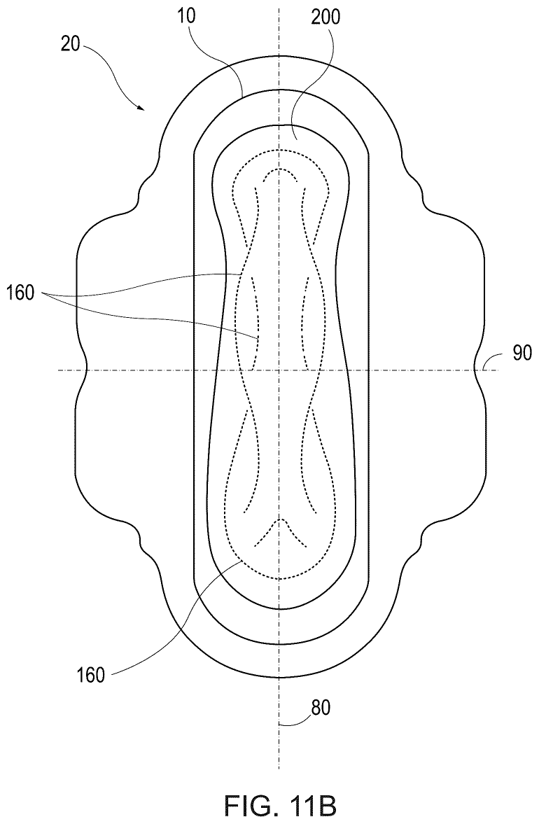

As shown in FIGS. 11A and 11B, the absorbent article may also comprise one or more flex bond channel regions 160, wherein the flex bond channel regions may be a continuous depression and/or a series of individually compressed, closely spaced embossments.

Suitable upper nonwoven layers may have a basis weight of from about 30 gsm to about 85 gsm, or from about 35 gsm to about 70 gsm, or from about 40 to about 60 gsm. The upper nonwoven layer may have a Tensile Stiffness of from about 0.1 N/mm to about 2.2 N/mm, or from about 0.3 N/mm to about 1.6 N/mm as measured according to the CD Cyclic Elongation to 3% Strain Method. The upper nonwoven layer may have a Strain to Break of greater than about 10%, or from about 10% to about 50%, or from about 20% to about 40%, as measured according to the Strain to Break Method. The upper nonwoven layer may have a Permanent Strain of about 0.005 to about 0.013 mm/mm, or from 0.005 to about 0.0090 mm/mm, as measured according to the CD Cyclic Elongation to 3% Strain Method.

Suitable lower nonwoven layers may have a basis weight of from about 7 gsm to about 40 gsm, or from about 10 gsm to about 35 gsm, or from about 15 gsm to about 20 gsm. The lower nonwoven layer may have a Tensile Stiffness of from about 0.2 N/mm to about 1.6 N/mm, as measured according to the CD Cyclic Elongation to 3% Strain Method. The lower nonwoven layer may have a Strain to Break of greater than about 10%, or from about 10% to about 50%, or from about 20% to about 40%, as measured according to the Strain to Break Method. The lower nonwoven layer may have a Permanent Strain of about 0.005 to about 0.013 mm/mm, as measured according to the CD Cyclic Elongation to 3% Strain Method.

The upper and/or lower nonwoven layers may comprise polymer fibers. Suitable upper and lower nonwoven fibers may be selected from PET (polyethylene terephthalate), PP (polypropylene), a BiCo (Bicomponent fiber) selected from PE/PP (PE sheath and PP core) and/or PE/PET (PE sheath PET core), PLA (polylactic acid), and combinations thereof. In some configurations, the upper and/or lower nonwoven layers may comprise recycled polymer resins, biodegradable polymers, biopolymers, biobased fibers, and combinations thereof.

Suitable upper nonwovens may comprise from about 60 to about 100%, or from about 70% to about 100% synthetic fibers, or from about 0 to about 40%, or from about 0 to about 30% regenerated cellulosic fibers, such as rayon and/or viscose.

The upper nonwoven layer may comprise fibers having a staple length of greater than about 10 mm, or greater than about 25 mm, or from about 10 mm to about 100 mm, or from about 20 mm to about 75 mm, or from about 25 mm to about 50 mm. The upper nonwoven layer may comprise fibers having a fiber diameter of from about 1.3 DTex to about 10.0 DTex, alternatively from about 1.3 DTex to about 6.0 DTex, alternatively from about 2.0 DTex to about 5.0 DTex. Without being limited by theory, it is believed that if the fibers of the upper nonwoven layer are less than about 1.3 Dtex, there may be insufficient air flow through the material during manufacturing.

In some configurations, the upper nonwoven layer may comprise a blend of staple fibers.

When the upper nonwoven layer comprises a blend of staple fibers, the blend of fibers preferably comprises 30% or less of fibers having a fiber diameter of 1.3 Dtex and/or 30% or less of fibers having a fiber diameter of 10.0 Dtex. In some configurations, the upper nonwoven layer may comprise fibers, wherein the fibers are a blend of staple fibers having an average fiber diameter of from about 2.0 DTex to about 8.0 DTex. Without being limited by theory, it is believed that fibers having an average fiber diameter of from about 2.0 Dtex to about 8.0 Dtex will help to enable sufficient air flow through the material during manufacturing of the absorbent core structure.

The lower nonwoven layer may comprise fibers having a staple length of greater than about 10 mm, or greater than about 25 mm, or from about 10 mm to about 100 mm, or from about 20 mm to about 75 mm, or from about 25 mm to about 50 mm. In some configurations, the lower nonwoven layer may comprise continuous fibers. The lower nonwoven layer may comprise fibers having a fiber diameter of from about 1.0 DTex to about 5.0 DTex, or from about 1.3 DTex to about 3.3 DTex, or from about 1.3 DTex to about 2.2 DTex, or from about 2.0 DTex to about 10.0 DTex. In some configurations, the lower nonwoven layer may comprise fibers, wherein the fibers are a blend of fibers having a fiber diameter of from about 0.1 DTex to about 6.0 DTex. In some configurations, upper nonwoven layer may comprise a blend of fibers, wherein at least a portion of the fibers have a diameter of from about 2.0 DTex to about 10.0 DTex and the lower nonwoven layer may comprise a blend of fibers, wherein at least a portion of the fibers have a diameter of from about 1.3 DTex to about 5.0 DTex. In some configurations, the upper nonwoven layer may comprise a blend of fibers, wherein at least a portion of the fibers have a diameter of from about 1.3 DTex to about 2.2 DTex and the lower nonwoven layer may comprise a blend of fibers, wherein the blend of fibers have a diameter of from about 1.0 DTex to about 5.0 DTex.

Suitable upper and/or lower nonwoven layer materials may bend and recover their original shape following the bending force. Flimsy or highly flexible materials readily bend at low peak force (load) and with low bending energy. Unsuitable materials, while readily bending, do not have sufficient recovery energy and so retain a deformed, bent state because of insufficient recovery energy. Suitable materials have sufficient energy to substantially recover their initial pre-bent state. The materials with sufficient bending recovery energy may be considered resilient upper and lower nonwoven layers. Particularly suitable upper nonwoven layers may have a Dry Recovery Energy of greater than about 0.03 N*mmm, or from about 0.03 N*mm to about 1 N*mm, or from about 0.04 N*mm to about 0.5 N*mm. Particularly suitable upper nonwoven layers may have a Dry Bending Energy of less than about 1.6 N*mm, or less than about 1.1 N*mm.

As noted above, the upper and lower nonwovens may include polymer fibers. Polymer fibers may be included to help provide structural integrity to the upper and lower nonwovens. The polymer fibers may help increase structural integrity of the upper and lower nonwovens in both a machine direction (MD) and in a cross-machine direction (CD), which may facilitate web manipulation during processing of the upper and lower nonwovens for incorporation into a pad.

Polymer fibers of any suitable composition may be selected. Some examples of suitable polymer fibers may include bi-component fibers comprising polyethylene (PE) and polyethylene terephthalate (PET) components or polyethylene terephthalate and co-polyethylene terephthalate components. The components of the bi-component fiber may be arranged in a sheath-core configuration, a side-by-side configuration, an eccentric sheath-core configuration, a trilobal arrangement, or any other desired configuration. In some configurations, the polymer fibers may include bi-component fibers having PE/PET components arranged in a concentric, sheath-core configuration, wherein the polyethylene component forms the sheath.

While other materials may be useful in creating a resilient structure, it is believed that the stiffness of a PET core component in a sheath-core fiber configuration may be useful for imparting resilience to the upper and lower nonwovens. In synergistic combination, a PE sheath component, having a lower melting temperature than the PET core component, may be utilized to provide inter-fiber melt/fusion bonding, effected via heat treatment of the precursor batt. This can help provide tensile strength to the web in both the MD and CD. Such inter-fiber bonds may serve to reduce fiber-to-fiber sliding, and thereby further contribute to imparting shape stability and resiliency to the material even when it is wetted.

Where a relatively higher weight fraction of polymer fibers is included, more connections within the structure may be created via heat treatment. However, too many connection points may impart greater stiffness to the upper and lower nonwovens than may be desirable. For this reason, selecting the weight fraction of the polymer fibers may involve prioritizing and balancing competing needs for stiffness and softness in the upper and lower nonwovens.

As noted above, the upper and lower nonwovens may additionally include polymer fibers which increase resiliency of the upper and lower nonwovens. The resilient polymer fibers may help the upper and lower nonwovens maintain permeability and compression recovery. In some configurations, the upper and lower nonwovens may comprise resilient polymer fibers having varying cross sections, e.g., round and hollow spiral, and/or may comprise resilient fibers having varying sizes.

The polymer fibers may be resilient and may be spun from any suitable thermoplastic resin, such as polypropylene (PP), polyethylene terephthalate (PET), or other suitable thermoplastics known in the art. The resilient polymer fibers may have any suitable structure or shape. For example, the resilient polymer fibers may be round or have other shapes, such as spiral, scalloped oval, trilobal, scalloped ribbon, and so forth. Further, the resilient polymer fibers may be solid, hollow, or multi-hollow. The resilient polymer fibers may be solid and round in shape. In other suitable examples, resilient polymer fibers may include polyester/co-extruded polyester fibers. In one particular example, PET fibers may be hollow in cross section and have a curl or spiral configuration along their lengths. Optionally, the resilient polymer fibers may be spiral-crimped or flat-crimped. The resilient polymer fibers may have an average crimp count of about 4 to about 12 crimps per inch (cpi), or about 4 to about 8 cpi, or about 5 to about 7 cpi, or about 9 to about 10 cpi. Particular non-limiting examples of resilient polymer fibers may be obtained from Wellman, Inc. (Ireland) under the trade designations H1311 and T5974. Other examples of suitable resilient polymer fibers are disclosed in U.S. Pat. No. 7,767,598.

The stiffening polymer fibers and resilient polymer fibers should be carefully selected. For example, while the constituent polymers forming the stiffening polymer fibers and the resilient polymer fibers may have similarities, resilient polymer fiber composition should be selected such that their constituents' melting temperature(s) is/are higher than that of the bondable components of the stiffening polymer fibers. Otherwise, during heat treatment, resilient polymer fibers could bond to stiffening polymer fibers and vice versa, and thereby an overly rigid structure. To avoid this risk where the stiffening polymer fibers include bicomponent fibers, e.g., core-sheath configuration fibers with a sheath component of relatively lower melting temperature at which fusion bonding will occur, the resilient polymer fibers may comprise the constituent chemistry of only the core, which may be a polymer having a relatively higher melting temperature.

Nonwoven performance can be impacted by a combination of the nonwoven fiber polymer choice, fiber properties and how the fibers are arranged or connected. Nonwoven selection may impact the absorbent article's ability to recover its shape following compression, bending and extension (stretching) forces present in-use with body motion. If the fibers are short fibers (less than about 10 mm) then the fibers are likely to irreversibly rearrange under extension and compressive forces. The rearranging (changing their orientation/state) of fibers in a fiber matrix dissipates the tensile (elongation) or compressive forces so that the energy used to affect the deformation is no longer available for recovery to the original shape. Longer fiber networks (typically greater than about 10 mm but less than about 100 mm) can absorb the tensile/compressive forces typical of bodily motions along the fiber length and across the structure. As a result, the absorbed forces are available to recover the structure to its original state. Longer fiber networks composed of finer fibers (less than about 15 to about 20 microns) more readily elongate and compress. As a result, the fluff/AGM structure can deform more readily (and to a higher degree) but the energy associated with these deformations is relatively small and insufficient to carry the structure back to its original state. Thicker fiber, such as greater than about 20 microns or 2.0 DTex to about 10 DTex, are both flexible under bodily forces but provide sufficient fiber and web recovery energy to return the structure to its original state.

The fiber arrangement in a long fiber network from a structural standpoint can impact the performance of the absorbent articles containing these nonwovens. Long fiber webs of thicker fibers are typically loftier than a conventional thin spunbond nonwoven web composed of continuous fine fibers that are closely spaced and physically bonded together. Creating a web of thicker fibers arranged in a more randomized orientation such as those that can be achieved via carding, hydro-entangling, and needling are able to elongate and compress, whereby the fibers only temporary adjust their arrangement (space between the fibers exist for these arrangements) and are able to carry/store the deformation forces and this energy is available for recovering the structural shape.

Additionally, finer (less than about 2.0 DTex) synthetic fibers such as BiCo and PP fibers commonly found in spunbond are closely spaced, relatively parallel aligned and closely bonded together. The bonded fibers within these spunbond webs are so interconnected (with closely spaced point bonds) that in tensile (elongation) the fibers at the polymer level are forced to stretch, resulting in polymer chains within the fiber permanently rearranging. As a result, the fibers themselves potentially remaining permanently elongated (permanently strained) and no longer able to recover to their initial state.

In some configurations, the polymer fibers in the upper nonwoven layer and the polymer fibers of the lower nonwoven layer may be different. In some configurations, the polymer fibers of the upper nonwoven layer and the polymer fibers of the lower nonwoven layer may be the same.

In some configurations, the upper nonwoven layer may be carded nonwoven. In some configurations, the upper nonwoven layer may be air through bonded or hydroentangled. In some configurations, the upper nonwoven layer is not a spunbond material.

Suitable nonwoven materials examples may include, but are not limited to, the following materials: (i) a 40 gsm carded resilient nonwoven material produced by Yanjan China (material code; ATB Z87G-40-90) which is a carded nonwoven composed of a blend of 60% 2 DTex and 40% 4 DTex BiCo (PE/PET) fibers. The fibers are bonded (ATB=Through ‘hot’ Air Bonded) to create a wet resilient network. The material basis weight is 40 gsm and its caliper (under 7 KPa) is about 0.9 mm. Without being limited by theory, it is believed that because of the presence of the 4 DTex BiCo fibers and the fiber-to-fiber bonded BiCo network, the material has a low Permanent Strain (less than about 0.013 mm/mm) and a sufficient Dry Recovery Energy (greater than about 0.03 N*mm) in the Wet and Dry CD Ultra Sensitive 3 Point Bending Method; (ii) a 55 gsm resilient spunlace material produced by Sandler Germany (material code: 53FC041001), which is a hydro-entangled nonwoven that is produced via a carding step (like the nonwoven described above) followed by hydro-entangling with an elevated drying step (as described in U.S. Patent Publication No. 2020/0315873 A1) that creates both an entangled and BiCo bonded resilient network. It comprises a fiber blend of 30% 10 DTex HS-PET, 50% 2.2 DTex BiCo (PE/PET), and 20% 1.3 DTex rayon. As such the material has a low Permanent Strain (less than about 0.013 mm/mm) and a sufficient Dry Recovery Energy (greater than about 0.03 N*mm) in the Wet and Dry CD Ultra Sensitive 3 Point Bending Method; and (iii) a 50 gsm resilient spunlace material produced by Sandler Germany (material code: 53FC041005 opt82), which is a hydro-entangled nonwoven that is produced via a carding step (like the nonwoven described above) followed by hydro-entangling with an elevated drying step (as described in U.S. Patent Publication No. 2020/0315873 A1) that creates both an entangled and BiCo bonded resilient network. It comprises a fiber blend of 60% 5.8 DTex BiCo (PE/PET), 20% 3.3 DTex tri-lobal ‘structural’ rayon, and 20% 1.3 DTex rayon. As such the material has a low Permanent Strain (less than about 0.013 mm/mm) and a sufficient Dry Recovery Energy (greater than about 0.03 N*mm) in the Wet and Dry CD Ultra Sensitive 3 Point Bending Method. While this material has 40% rayon that can soften when wet, the use of structural tri-lobal rayon fibers helps structural stability in the wet state.

In combination with adjustment of pore size, volume, and number via selection of appropriate fiber size, basis weight, and extent of consolidation, the manufacturer may wish to select fiber constituents for having particular surface chemistry(ies), e.g., fibers with hydrophobic surfaces, hydrophilic surfaces, or a blend of differing fibers and/or z-direction stratification or gradient thereof. Fibers having hydrophilic surfaces will tend to attract and move aqueous components of menstrual fluid there along in a manner conducive to wicking and rapid fluid acquisition following discharge. At the same time, however, a predominance of hydrophilic fibers surfaces within the topsheet may increase a tendency of the topsheet to reacquire fluid from absorbent components beneath (rewet), which can cause an undesirable wet feel for the user. On the other hand, fibers having hydrophobic surfaces will tend to repel aqueous components of menstrual fluid and/or resist movement of fluid along their surfaces, thereby tending to resist wicking—but also to resist rewetting. The manufacturer may wish to seek an appropriate balance in selecting constituent fibers having hydrophilic surfaces, fibers having hydrophobic surfaces, or a blend and/or z-direction stratification thereof, in combination with fiber size, fiber consolidation level, and resulting topsheet pore size, volume and number, for any particular product design.

The inner core layer is produced in an airlaying process. Streams of cellulose and superabsorbent particles are carried on a fast moving airstream and deposited into a three dimensionally shaped pocket on a rotating forming drum with a vacuum below to draw the cellulose and superabsorbent particles into the pocket in a laydown station. This shaped pocket provides the actual physical shape of the absorbent core structure. The upper or lower nonwoven may be first introduced onto the forming drum and under the vacuum the upper or lower nonwoven are drawn into the 3-dimensional pocket shape. In this case, the cellulose and superabsorbent particle material stream is deposited on the upper (or lower nonwoven material) directly in the forming station. Prior to entering the forming station, the nonwoven is coated with an adhesive to provide a stronger connection of the cellulose and superabsorbent particles to the nonwoven layer.

Particularly suitable adhesives can include high wet strength adhesives such as a Technomelt DM9036U available from Henkel (Germany). On exiting the laydown section, the second remaining nonwoven layer is combined with the nonwoven carrying the cellulose and superabsorbent particle layer exiting the laydown section. This second remaining nonwoven (either upper or lower nonwoven depending on what nonwoven is run through the laydown section) is precoated with adhesive to enable a perimeter seal and to better integrate the cellulose and superabsorbent particles without hindering the flow of liquid into the cellulose and superabsorbent particle matrix. In another approach, a nonwoven is not first introduced into the forming station and the cellulose and superabsorbent particle mass is held on the forming drum under vacuum until it is ejected onto either the upper or lower nonwoven layer that has an adhesive applied as detailed above and then sealed with the second remaining nonwoven to create the absorbent core structure. The width of the upper and lower nonwoven webs is typically chosen to be wider than the maximum width of the shaped cellulose and superabsorbent particle matrix so as to enable an effective perimeter seal where the two nonwovens connect, at least on the left and right most sides of the absorbent core structure.

The inner core layer may comprise any of a wide variety of liquid-absorbent materials commonly used in absorbent articles, such as comminuted wood pulp, which is generally referred to as airfelt. One suitable absorbent core material is an airfelt material which is available from Weyerhaeuser Company, Washington, USA, under Code No. FR516. Examples of other suitable liquid-absorbent materials for use in the absorbent core may include creped cellulose wadding; meltblown polymers including coform; chemically stiffened, modified or cross-linked cellulosic fibers; synthetic fibers such as crimped polyester fibers; peat moss; cotton; bamboo; eucalyptus; absorbent polymer materials; or any equivalent material or combinations of materials, or mixtures of these.

Absorbent polymer materials for use in absorbent articles typically comprise water-insoluble, water-swellable, hydrogel-forming crosslinked absorbent polymers which are capable of absorbing large quantities of liquids and of retaining such absorbed liquids under moderate pressure.

The absorbent polymer material for the absorbent cores according to the present disclosure may comprise superabsorbent particles, also known as “superabsorbent materials” or as “absorbent gelling materials”. Absorbent polymer materials, typically in particle form, may be selected among polyacrylates and polyacrylate based materials, such as for example partially neutralized, crosslinked polyacrylates. The term “particles” refers to granules, fibers, flakes, spheres, powders, platelets and other shapes and forms known to persons skilled in the art of superabsorbent particles. In some aspects, the superabsorbent particles may be in the shape of fibers, i.e., elongated, acicular superabsorbent particles.

In some configurations, the inner core layer may comprise cellulosic fibers and superabsorbent particles. In some configurations, the inner core layer may comprise from about 50% to about 85% cellulosic fiber, or from about 55% to about 80%, or from about 60% to about 75%, all by weight of the inner core layer. The inner core layer may comprise from about 15% to about 50% superabsorbent particles, or from about 20% to about 50%, or from about 25% to about 40%, or from about 30% to about 35%, all by weight of the inner core layer. In some configurations, the inner core layer may comprise from about 125 gsm to about 350 gsm cellulosic fiber. In some configurations, the inner core layer may comprise from about 20 gsm to about 125 gsm superabsorbent particles.

In some configurations, the inner core layer may comprise from about 50% to about 85% cellulosic fiber and from about 15% to about 50% superabsorbent particles. The absorbent core structure may have an average density of between about 0.045 g/cm³ and about 0.15 g/cm³, and/or between 0.045 g/cm³ and 0.12 g/cm³. The absorbent article may have an average density of between about 0.045 g/cm³ and about 0.16 g/cm³.

The absorbent core structures may compress and substantially recover their original shape following the compression step. Suitable absorbent core structures require a low force to compress (less resistance) and the structure is able to substantially recover its shape as the user, in a cyclic fashion, compresses and releases the compressive force with various body movements. To achieve this, the structure sustains sufficient recovery energy following multiple cyclic compressions. Without sufficient recovery energy the structure may remain in a compressed bunched state with insufficient force (stored energy) to recover.

The absorbent article 20 may be resilient and conformable and may deliver a superior in-use experience without substantially bunching and/or compressing. The absorbent article may be exposed to bodily forces and may substantially recover to its original state. The absorbent article may have a CD Dry Modulus of between about 0.07 and 0.30 N/mm² as measured in the Wet and Dry CD and MD 3 Point Bend Method, or from about 0.10 to about 0.25 N/mm², or from about 0.10 to about 0.20 N/mm².

The absorbent article may have a of Dry Caliper between about 2.0 mm and about 6.0 mm, or from about 2.0 mm and about 4.5 mm, or from about 2.5 mm to about 4.0 mm, as measured according to the Wet and Dry CD and MD 3-Point Method. In some configurations, the absorbent article may have a CD Dry Modulus of between about 0.07 and 0.30 N/mm² and a Dry Caliper between about 2.0 mm and about 4.5 mm as measured according to the Wet and Dry CD and MD 3-Point Method, or a CD Dry Modulus of between from about 0.10 to about 0.25 N/mm² and a Dry Caliper of from about 2.5 mm to about 4.0 mm. The absorbent article may have a CD Dry Bending Stiffness of between about 7.0 to about 30.0 N*mm² as measured in the Wet and Dry CD and MD 3 Point Bend Method, or about 10.0 and about 25.0 N*mm², or about 10.0 to about 20.0 N*mm², or about 13.0 to about 20.0 N*mm².

The absorbent article may have a 5^{th} Cycle Wet Energy of Recovery of from about 1.0 to 3.5 N*mm, or about 1.5 to about 3.0 N*mm, or about 1.5 to about 2.8 N*mm. Particularly suitable absorbent articles may have a 5^{th} Cycle Wet Energy of Recovery of between about 1.0 and 3.5 N*mm and a 5^{th} Cycle Wet % Recovery of from about 29% to about 40%.

### Topsheet

Topsheet 110 may be formed of any suitable nonwoven web or formed film material. Referring back to the figures, the topsheet 110 is positioned adjacent a wearer-facing surface of the absorbent article 20 and may be joined thereto and to the backsheet 130 by any suitable attachment or bonding method. The topsheet 110 and the backsheet 130 may be joined directly to each other in the peripheral regions outside the perimeter of the absorbent core structure and may be indirectly joined by directly joining them respectively to wearer-facing and outward-facing surfaces of the absorbent article or additional optional layers included with the absorbent article.

The absorbent article 20 may have any known or otherwise effective topsheet 110, such as one which is compliant, soft feeling, and non-irritating to the wearer's skin. A suitable topsheet material will include a liquid pervious material that is comfortable when in contact with the wearer's skin and permits discharged menstrual fluid to rapidly penetrate through it. Some suitable examples of topsheet materials include films, nonwovens, laminate structures including film/nonwoven layers, film/film layers, and nonwoven/nonwoven layers.

Nonlimiting examples of nonwoven web materials that may be suitable for use to form the topsheet 110 include fibrous materials made from natural fibers, modified natural fibers, synthetic fibers, or combinations thereof. Some suitable examples are described in U.S. Pat. Nos. 4,950,264; 4,988,344; 4,988,345; 3,978,185; 7,785,690; 7,838,099; 5,792,404; and 5,665,452.

The topsheet 110 may be compliant, soft feeling, and non-irritating to the wearer's skin. Further, the topsheet 110 may be liquid pervious permitting liquids (e.g., urine, menses) to readily penetrate through its thickness. Some suitable examples of topsheet materials include films, nonwovens, laminate structures including film/nonwoven layers, film/film layers, and nonwoven/nonwoven layers. Other exemplary topsheet materials and designs are disclosed in U.S. Patent Application Publication Nos. 2016/0129661, 2016/0167334, and 2016/0278986.

In some examples, the topsheet 110 may include tufts as described in U.S. Pat. Nos. 8,728,049; 7,553,532; 7,172,801; 8,440,286; 7,648,752; and 7,410,683. The topsheet 20 may have a pattern of discrete hair-like fibrils as described in U.S. Pat. No. 7,655,176 or U.S. Pat. No. 7,402,723. Additional examples of suitable topsheet materials include those described in U.S. Pat. Nos. 8,614,365; 8,704,036; 6,025,535; and US Patent Publication No. 2015/041640. Another suitable topsheet may be formed from a three-dimensional substrate as detailed in US 2017/0258647. The topsheet may have one or more layers, as described in US Patent Publication Nos. 2016/0167334; 2016/0166443; and 2017/0258651.

In some examples a topsheet 110 may be formed of a nonwoven web material of a spunbond web including single-component continuous fibers, or alternatively, bi-component or multi-component fibers, or a blend of single-component fibers spun of differing polymer resins, or any combination thereof. The topsheet may also be a formed nonwoven topsheet as disclosed in US Patent Publication No. 2019/0380887.

In order to ensure that fluid contacting the top (wearer-facing) surface of a topsheet will move suitably rapidly in a z-direction to the bottom (outward-facing) surface of the topsheet where it can be drawn into the absorbent article, it may be important to ensure that the nonwoven web material forming the topsheet has an appropriate weight/volume density, reflecting suitable presence of interstitial passageways (sometimes known as “pores”) among and between the constituent fibers, through which fluid may move within the nonwoven material. In some circumstances a nonwoven material with fibers that are consolidated too densely may have insufficient numbers and/or volumes and/or sizes of pores, and the nonwoven will obstruct rather than facilitate rapid downward z-direction fluid movement. On the other hand, a nonwoven with fibers that are too large and/or not consolidated enough to provide a certain level of opacity (for purposes of concealing absorbed fluid in the layers beneath) and a substantial appearance may be negatively perceived by users.

The caliper of the topsheet material may be controlled, to balance competing needs for opacity and loft (which call for a higher caliper) vs. a limitation on the z-direction distance that discharged fluid travels through the topsheet from the wearer-facing surface to the outward-facing surface to reach the absorbent core structure below. Thus, it may be desired that the manufacture of the topsheet material be controlled to produce a topsheet material having a caliper of from about 0.20 mm to about 1.0 mm, or from about 0.25 mm to about 0.80 mm, or from about 0.30 mm to about 0.60 mm.

In some configurations, the absorbent article may not comprise a discrete topsheet and the upper nonwoven layer may function as the topsheet.

### Secondary Topsheet (STS)

An STS layer may be included, in some circumstances, between the topsheet and the absorbent core structure to enable the absorbent core structure to readily receive a sudden discharge of fluid, and after receipt, to wick it along x- and y-directions to distribute it across the underlying absorbent core structure.

If included, an STS may be a nonwoven fibrous structure which may include cellulosic fibers, non-cellulosic fibers (e.g., fibers spun from polymer resin(s)), or a blend thereof. To accommodate the folding and lateral gathering of the absorbent article 20, and of the absorbent core structure 10, as described herein, the STS may be formed of a material that is relatively pliable (i.e., has relatively low bending stiffness).

A number of particular examples of suitable STS compositions and structures, as well as combinations thereof with suitable topsheet compositions and structures, are further described in U.S. Apps. Ser. Nos. 16/831,862; 16/831,854; 16/832,270; 16/831,865; 16/831,868; 16/831,870; and Ser. No. 16/831,879; and U.S. Provisional Apps. Ser. Nos. 63/086,610 and 63/086,701. Additional suitable examples are described in U.S. Pat. No. 9,504,613; WO 2012/040315; and US 2019/0021917.

In some configurations, the absorbent article may be free of a secondary topsheet.

### Backsheet

The backsheet 130 may be positioned beneath or subjacent an outward-facing surface of the absorbent core structure 10 and may be joined thereto by any suitable attachment methods. For example, the backsheet 130 may be secured to the absorbent core structure 10 by a uniform continuous layer of adhesive, a patterned layer of adhesive, or an array of separate lines, spirals, or spots of adhesive. Alternatively, the attachment method may include heat bonds, pressure bonds, ultrasonic bonds, dynamic mechanical bonds, or any other suitable attachment mechanisms or combinations thereof. In other examples, it is contemplated that the absorbent core structure 10 is not joined directly to the backsheet 130.

The backsheet 130 may be impermeable or substantially impermeable by aqueous liquids (e.g., urine, menstrual fluid) and may be manufactured from a thin plastic film, although other flexible liquid impermeable materials may also be used. As used herein, the term “flexible” refers to materials which are compliant and will readily conform to the general shape and contours of the human body. The backsheet 130 may prevent, or at least substantially inhibit, fluids absorbed and contained within the absorbent core structure 10 from escaping and reaching articles of the wearer's clothing which may contact the absorbent article 20, such as underpants and outer clothing.

However, in some instances, the backsheet 130 may be made and/or adapted to permit vapor to escape from the absorbent core structure 10 (i.e., the backsheet is made to be breathable), while in other instances the backsheet 130 may be made so as not to permit vapors to escape (i.e., it is made to be non-breathable). Thus, the backsheet 130 may comprise a polymeric film such as thermoplastic films of polyethylene or polypropylene. A suitable material for the backsheet 130 is a thermoplastic film having a thickness of from about 0.012 mm (0.5 mil) to about 0.051 mm (2.0 mils), for example. Any suitable backsheet known in the art may be utilized with the present invention.

Some suitable examples of materials suitable for forming a backsheet are described in U.S. Pat. Nos. 5,885,265; 4,342,314; and 4,463,045. Suitable single layer breathable backsheets for use herein include those described for example in GB A 2184 389; GB A 2184 390; GB A 2184 391; U.S. Pat. Nos. 4,591,523; 3,989,867; 3,156,242; WO 97/24097; U.S. Pat. Nos. 6,623,464; 6,664,439; and 6,436,508.

The backsheet 130 may have two layers: a first layer comprising a vapor permeable aperture-formed film layer and a second layer comprising a breathable microporous film layer, as described in U.S. Pat. No. 6,462,251. Other suitable examples of dual or multi-layer breathable backsheets for use herein include those described in U.S. Pat. Nos. 3,881,489; 4,341,216; 4,713,068; 4,818,600; EP 203 821; EP 710 471; EP 710 472; and EP 0 793 952.

### Other Features

In some configurations, the absorbent article 20 may comprise a panty fastening component, such as a panty fastening adhesive or components of a hook and-loop fastening system (such as VELCRO®).

In some configurations, the absorbent article 20 may be provided with a panty fastening adhesive disposed on the garment-facing side of the backsheet 130 in order to provide a mechanism for the user to adhere the absorbent article to the inside of her underpants in the crotch region thereof. The panty fastening adhesive may comprise any adhesive or glue used in the art for such purposes. These adhesives typically are pressure sensitive and remain tacky well below their application temperature. In some configurations, the panty fastening adhesive may be a pressure sensitive hot melt adhesive. The panty fastening adhesive may be applied in a pattern such as described in U.S. Patent Publication No 2020/0281782A1. When the absorbent article 20 is packaged for shipping, handling and storage prior to use, panty fastening adhesive may be covered by a protective cover (not shown) such as a silicone coated release paper, a silicone coated plastic film, or any other easily removable cover. The protective cover can be provided as a single piece or in a multitude of pieces, e.g., to cover individual adhesive areas such as on the backsheet and/or on the wings. The protective cover may cover/shield the adhesive deposits from contact with other surfaces until the user is ready to remove the protective cover and place the absorbent article in her underpants for wear/use. The protective cover may also function as an individualized packaging for the article or provide a disposal function as known in the art. Any commercially available release paper or film may be used. Suitable examples include BL 30 MG-A SILOX EI/O, BL 30 MG-A SILOX 4 P/O available from Akrosil Corporation, and M&W films available from Gronau in Germany, under the code X-5432. In some configurations, the absorbent article may be packaged in a bi-folded or tri-folded state.

In some configurations, the absorbent article 20 may include opposing wing portions 140, 150 on each side, extending laterally beyond longitudinal edges of the absorbent portions of the absorbent article by a comparatively greater width dimension than that of the forward and rearward portions of the absorbent article. Wings are currently commonly provided with feminine hygiene absorbent articles. As provided, they typically have deposits of adhesive applied to their outward-facing surfaces (surface are outward-facing prior to placement of the absorbent article within the user's underwear and application of the wings). The wing portions may also include deposits of adhesive as described above, which enable the user to wrap the wing portions through the leg openings of the underpants and around the inside edges thereof, and adhere the wing portions to the outward-facing surface/underside of the underpants in the crotch region, providing supplemental holding support for the absorbent article and helping guard the underpants proximate the leg edges thereof against soiling.

### Packages

Typically, products such as absorbent articles are not sold individually, but rather are sold in packages containing a plurality of absorbent articles, such as in packages of from about 5 to about 30 absorbent articles. The absorbent articles of the present disclosure may be placed into packages. The packages may comprise polymeric films, paper material, carton board, and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, users and caregivers can easily handle and store the packages, especially in bathrooms and other areas with limited space, while also providing distribution savings to manufacturers owing to the size of the packages.

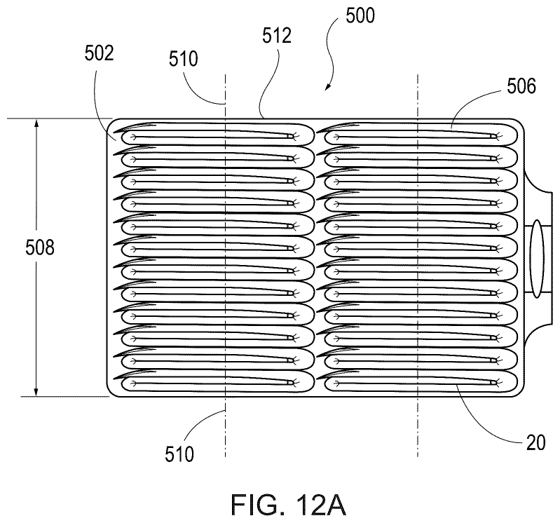

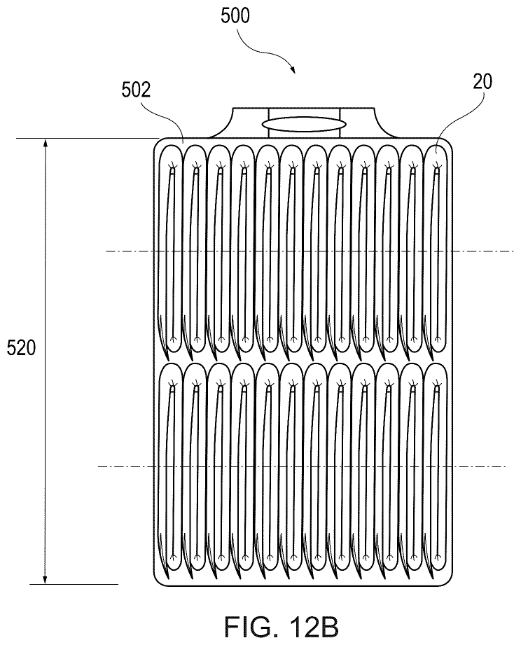

In some configurations, the absorbent articles may be packaged in a poly bag. In some configurations, the package may be a plastic “shrink-wrap” container. As shown in FIG. 12A, package 500 is a poly bag. Package 500 has an interior space 502, an exterior surface 512 and a height, width, and depth dimension. Package 500 may be any shape known in the art. For example, the package may have a polyhedral shape defining or forming a polyhedral enclosure. Interior 502 defines an interior space for containing absorbent articles 20. In some configurations, the absorbent articles may all be identical to one another.

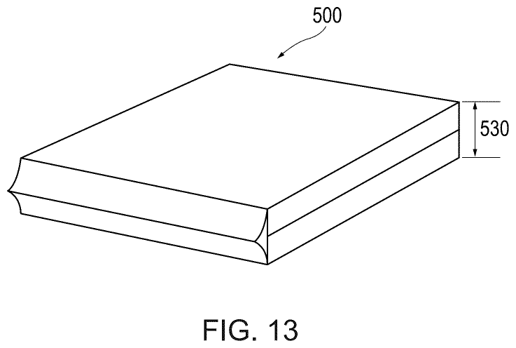

The absorbent articles 20 may be arranged to form a stack 506 within interior 502 of package 500. The articles may be stacked in any direction. As used herein, the term “stack” means an orderly pile. For example, the articles may be stacked vertically, horizontally, or at any angle inside the interior of the package. As shown in FIG. 12A, package 500 has a filled bag width (FBW) 508 that is defined as the maximum distance between the two highest bulging points along the same compression stack axis 510 of the package. As illustrated in FIG. 12B, package 500 has a filled bag height (FBH) 520 that is defined as the maximum distance between the bottom panel and highest point of the top panel. As illustrated in FIG. 13, package 500 has a filled bag depth (FBD) 530 is defined as the maximum distance between the front and back panels of the package 500. Absorbent articles according to the present disclosure can be bi-folded, tri-folded, rolled, unfolded, or any other suitable configuration for packaging of absorbent articles. The package 500 may also include a mechanism or means for accessing the interior space, for example, a gusset, a line of perforations, tabs, adhesive openings or any other means known in the art.

Package 500 may be composed of different materials or may be composed of substantially the same type of material. Package 500 may be composed of one layer or a laminate. The material can comprise a blown or cast film in a blend of low density polyethylene and linear low density polyethylene, metallocenes, ethylene vinyl acetate, surlyn, polyethylene terephtalate, biaxially oriented polypropylene, and/or nylon.

In some configurations, package 500 may comprise polymeric films comprising recycled material, such as about 20% to about 100%, about 30% to about 90%, about 30% to about 80%, about 40% to about 60%, or about 50% recycled material. The recycled material may comprise post-industrial recycled material (PIR) and/or post-consumer recycled material (PCR). In some instances, the polymeric films used for the packages may comprise two outer layers and one or more inner layers. The one or more inner layers may comprise the recycled material or may comprise more recycled material than the outer layers. The recycled material may comprise recycled polyethylene. The recycled material may comprise recycled polyethylene PIR from trim from the packaging operation.

The package material may comprise paper, paper based material, paper with one or more barrier layers, or a paper/film laminate. The package material may be in the range of about 50 gsm to about 100 gsm or about 70 gsm to about 90 gsm and the one or more barrier layers may be in the range of about 3 gsm to about 15 gsm. The paper based package material with or without one or more barrier layers may exhibit a machine direction tensile strength of at least 5.0 kN/m, a machine direction stretch of at least 3 percent, a cross-machine direction tensile strength of at least 3 kN/m, and a cross-direction stretch at break of at least 4 percent, each as determined via ISO 1924-3.

The paper based package material or paper based package material comprising a barrier layer or film may be recyclable or recyclable in normal paper recycling operations. The recyclability extent of the paper based package may be determined via recyclable percentage. The paper based package of the present disclosure may exhibit recyclable percentages of 70 percent or greater, 80 percent or greater, or 90 percent or greater. The paper based package of the present disclosure may have a recyclable percentage of between 70 percent to about 99.9 percent, between about 80 percent to about 99.9 percent, or between about 90 percent to about 99.9 percent. In one example, the package material of the present disclosure may exhibit a recyclable percentage of from about 95 percent to about 99.9 percent, from about 97 percent to about 99.9 percent, or from about 98 percent to about 99.9 percent. The recyclable percentage of the paper based package may be determined via test PTS-RH:021/97 (Draft October 2019) under category II, as performed by Papiertechnische Stiftung located at Pirnaer Strasse 37, 01809 Heidenau, Germany. In another instance, the paper based packages of the present disclosure may exhibit an overall “pass” test outcome as determined by PTS-RH:021/97 (Draft October 2019) under category II method. Any of the paper based packages may have opening features, such as lines of perforation, and may also have handles.

It was surprisingly found that the absorbent articles described herein may be compressed and held in packages for an extended time period and still substantially recover pre-compression caliper. For example, absorbent products according to the present disclosure may have an in-bag compression of about 40% or less, or from about 5% to about 35%, or from about 15% to about 30%, without adversely affecting flexibility, conformability, or fluid handling performance of the absorbent article. “In-Bag Compression” as used herein is one minus the height of a stack of 10 absorbent articles in millimeters, measured while under compression within a package, divided by the height of a stack of 10 absorbent articles of the same type before compression, multiplied by 100, and reported as a percentage.

In some configurations, the absorbent products may comprise absorbent articles having an Average In-Bag Pad Caliper of about 2.0 mm to about 5.5 mm, or from about 2.5 mm to about 4.0 mm, or from about 2.75 mm to about 3.5 mm, as measured according to the In Bag Compression Recovery Method.

In some configurations, the absorbent products may comprise absorbent articles having an Average Out-of-Bag Pad Caliper after 2 minutes from removal from the package of from about 2.0 mm to about 6.0 mm, or from about 3.0 mm to about 5.0 mm, as measured according to the In Bag Compression Recovery Method. In some configurations, the absorbent products may comprise absorbent articles having an Average Out-of-Bag Pad Caliper after 4 hours from removal from the package of from about 2.0 mm to about 6.0 mm, or from about 3.0 mm to about 5.0 mm, as measured according to the In Bag Compression Recovery Method.

In some configurations, the absorbent products may comprise absorbent articles having a % Caliper Recovery after 2 minutes from removal from the package of at least 4%, or at least 10%, or at least 15%, as measured according to the In Bag Compression Recovery Method. In some configurations, the absorbent products may comprise absorbent articles having a % Caliper Recovery after 2 minutes from removal from the package of from about 3% to about 35%, or from about 10% to about 30%, or from about 12% to about 25%, as measured according to the In Bag Compression Recovery Method.

In some configurations, the absorbent products may comprise absorbent articles having a % Caliper Recovery after 4 hours from removal from the package of at least 4%, or at least 10%, or at least 15%, as measured according to the In Bag Compression Recovery Method. In some configurations, the absorbent products may comprise absorbent articles having a % Caliper Recovery after 4 hours from removal from the package of from about 4% to about 35%, or from about 10% to about 30%, or from about 15% to about 30%, as measured according to the In Bag Compression Recovery Method.

In some configurations, the absorbent products may comprise absorbent articles having an Average In-Bag Pad Density of about 0.20 g/cm³ or less, or from about 0.07 g/cm³ to about 0.17 g/cm³, or from about 0.10 g/cm³ to about 0.15 g/cm³, as measured according to the In Bag Compression Recovery Method.

In some configurations, the absorbent products may comprise absorbent articles having an Average Out-of-Bag Pad Density after 2 minutes from removal from the package of from about 0.06 g/cm³ to about 0.16 g/cm³, as measured according to the In Bag Compression Recovery Method. In some configurations, the absorbent products may comprise absorbent articles having an Average Out-of-Bag Pad Density after 4 hours from removal from the package of from about 0.06 g/cm³ to about 0.16 g/cm³, as measured according to the In Bag Compression Recovery Method.

The present disclosure also relates to a process for packaging a plurality of disposable absorbent articles, such as feminine hygiene pads. The process may comprise the steps of:
a. providing a plurality of disposable absorbent articles, each of the disposable absorbent articles comprising an absorbent core structure disposed between a topsheet and a backsheet, the absorbent core structure comprising an upper nonwoven layer comprising polymer fibers and having a basis weight of from about 30 gsm to about 85 gsm, a lower nonwoven layer comprising polymer fibers and having a basis weight of from about 7 gsm to about 40 gsm, and an inner core layer comprising cellulosic fibers and superabsorbent particles, wherein at least a portion of the inner core layer is disposed between the upper nonwoven layer and the lower nonwoven layer;
b. folding each disposable absorbent article of the plurality of disposable absorbent articles to form a plurality of folded disposable absorbent articles;
c. arranging the plurality of folded disposable absorbent articles to form a stack of folded disposable absorbent articles;
d. compressing the stack of folded disposable absorbent articles along a compression axis to form a compressed stack of folded disposable absorbent articles;
e. placing the compressed stack of folded disposable absorbent articles in an interior space of a package, wherein the compressed stack of folded disposable absorbent articles is placed in the interior space of the package such that the compression axis of the stack of folded disposable absorbent articles is oriented substantially along the width dimension of the package; and
f. closing the package such that the folded disposable absorbent articles exhibit an Average In-Bag Folded Caliper of less than 18 mm, or from about 7.0 to about 15.0 mm, or from about 8.0 mm to about 13.0 mm, and such that upon removal from the package, the disposable feminine hygiene pads exhibit a % Caliper Recovery at 2 minutes of at least 4%, as measured according to the In Bag Compression Recovery Method.

In some configurations, the plurality of disposable absorbent articles may not be folded and may be arranged to form a stack of substantially flat disposable absorbent articles. In such configurations the disposable absorbent articles may exhibit an Average In-Bag Pad Caliper of from about 2.5 mm to about 5.5 mm.

### Test Methods

### Layers of Interest

For any of the methods below in which all the component layers of an article will not be tested, the layers of interest may be separated using cryo-spray as needed from layers which will not be tested.

### Strain to Break Method

The force versus displacement behavior of a sample is measured on a universal constant rate of extension test frame (a suitable instrument is the MTS Alliance using TestSuite Software, as available from MTS Systems Corp., Eden Prairie, MN, or equivalent) equipped with a load cell for which the forces measured are within 1% to 99% of the limit of the cell. The sample is subjected to tensile elongation at a constant rate (mm/sec) until it breaks, and the percent strain to break is measured. All testing is performed in a room controlled at 23° C.±3 C.° and 50%±2% relative humidity and test samples are conditioned in this environment for at least 2 hours prior to testing.

The fixtures used to grip the test specimen are lightweight (<80 grams), vise action clamps with half cylinder steel versus rubber coated steel grip faces that are at least 40 mm wide. The fixtures are installed on the universal test frame and mounted such that they are horizontally and vertically aligned with one another.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is prepared as follows. The test specimen is cut from an area on the test material that is free of any folds or wrinkles. The test specimen is 100 mm long (parallel to the lateral axis, or intended lateral axis of the article) and 25.4 mm wide (parallel to the longitudinal axis, or intended longitudinal axis of the article). In like fashion, five replicate test specimens are prepared.

Prepare the universal test frame as follows. Set the initial grip to grip separation distance to a nominal gage length of 80 mm, then zero the crosshead. Program the test frame to move the grips closer together by an intentional slack of 1 mm to ensure no pretension force exists on the test specimen at the onset of the test. (During this motion, the specimen will become slack between the grips.) Next, the grips will move apart at a slack speed of 1 mm/s until the slack preload of 0.05 N is exceeded. (At this point, the crosshead position signal is used to compute the sample slack, the adjusted gage length, and the strain is defined at zero, 0.0). The grips will then move apart at a speed of 1 mm/s until the sample breaks or the extension limit of the instrument is exceeded.

The test is executed by inserting the test specimen into the grips such that the long axis of the specimen is parallel and centered with the motion of the crosshead. Start the test and continuously collect force (“load”) and displacement data at a data acquisition rate of 100 Hz.

Construct a graph of load (N) versus displacement (mm). Determine the peak load from the curve, then determine the break sensitivity as follows. Determine the crosshead position at which the load signal decreases by 75% after the peak load is reached, and record as specimen final length (Lf) to the nearest 0.01 mm. The initial length of the specimen is defined by the crosshead position when the slack preload of 0.05 N is exceeded, and this value is recorded as specimen initial length (Li) to the nearest 0.01 mm. Calculate the percent strain to break as follows, and record to the nearest 1 percent.

$$\% Strain to Break = ( ( Lf - Li ) / Li ) * 100$$

In like fashion, the procedure is repeated for all five replicate test specimens. The arithmetic mean of % strain to break among the five replicate test specimens is calculated and reported as % Strain to Break to the nearest 1 percent.

### Wet and Dry CD and MD 3 Point Bend Method

The bending properties of an absorbent article test sample are measured on a universal constant rate of extension test frame (a suitable instrument is the MTS Alliance using TestSuite Software, as available from MTS Systems Corp., Eden Prairie, MN, or equivalent) equipped with a load cell for which the forces measured are within 1% to 99% of the limit of the cell. The test is executed on dry test specimens as well as wet test specimens. The intention of this method is to mimic deformation created in the x-y plane by a wearer of an absorbent article during normal use. All testing is performed in a room controlled at 23° C.±3 C and 50%±2% relative humidity.

The bottom stationary fixture consists of two cylindrical bars 3.175 mm in diameter by 110 mm in length, made of polished stainless steel each mounted on each end with frictionless roller bearings. These 2 bars are mounted horizontally, aligned front to back and parallel to each other, with top radii of the bars vertically aligned and are free to rotate around the diameter of the cylinder by the frictionless bearings. Furthermore, the fixture allows for the two bars to be moved horizontally away from each other on a track so that a gap can be set between them while maintaining their orientation. The top fixture consists of a third cylinder bar also 3.175 mm in diameter by 110 mm in length, made of polished stainless steel mounted on each end with frictionless roller bearings. When in place the bar of the top fixture is parallel to and aligned front to back with the bars of the bottom fixture and is centered between the bars if the bottom fixture. Both fixtures include an integral adapter appropriate to fit the respective position on the universal test frame and lock into position such that the bars are orthogonal to the motion of the crossbeam of the test frame.

Set the gap (“Span”) between the bars of the lower fixture to 25 mm±0.5 mm (center of bar to center of bar) with the upper bar centered at the midpoint between the lower bars. Set the gage (bottom of top bar to top of lower bars) to 1.0 cm.

The thickness (“caliper”) of the test specimen is measured using a manually-operated micrometer equipped with a pressure foot capable of exerting a steady pressure of 0.1 psi+0.01 psi. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter no greater than 25.4 mm. The test specimen is supported by a horizontal flat reference platform that is larger than and parallel to the surface of the pressure foot. Zero the micrometer against the horizontal flat reference platform. Place the test specimen onto the platform, centered beneath the pressure foot. The pressure foot is lowered by hand with a descent rate of 3±1 mm/s until the full weight of the pressure is exerted onto the specimen. After 5 seconds elapse, the thickness is recorded as caliper to the nearest 0.01 mm.

The test fluid used to dose the wet test specimens is prepared by adding 100.0 grams of sodium chloride (reagent grade, any convenient source) to 900 grams of deionized water in a 1-liter Erlenmeyer flask. Agitate until the sodium chloride is completely dissolved.

The absorbent article samples are conditioned at 23° C.±3 C and 50%±2% relative humidity two hours prior to testing. Dry test specimens are taken from an area of the sample that is free from any seams and residua of folds or wrinkles, and ideally from the center of absorbent article (intersection of longitudinal and lateral midlines). The dry specimens are prepared for MD (machine direction) bending by cutting them to a width of 50.8 mm along the CD (cross direction; parallel to the lateral axis of the sample) and a length of 50.8 mm along the MD (parallel to the longitudinal axis of the sample), maintaining their orientation after they are cut, and marking the body-facing surface (or the surface intended to face the body of a finished article). The dry specimens are prepared for CD (machine direction) bending by cutting them to a width of 50.8 mm along the MD (cross direction; parallel to the lateral axis of the sample) and a length of 50.8 mm along the CD (parallel to the longitudinal axis of the sample), maintaining their orientation after they are cut, and marking the body-facing surface (or the surface intended to face the body of a finished article). Measure the thickness of the test specimen, as described herein, and record as dry specimen caliper to the nearest 0.01 mm. Now measure the mass of the test specimen and record as dry mass to the nearest 0.001 grams. Calculate the basis weight of the specimen by dividing the mass (g) by the area (0.002581 m²) and record as dry specimen basis weight to the nearest 0.01 g/m². Calculate the bulk density of the specimen by dividing the specimen basis weight (g/m²) by the specimen thickness (mm), then dividing the quotient by 1000, and record as dry specimen density to the nearest 0.01 g/cm³. In like fashion, five replicate dry test specimens are prepared.

Wet test specimens are initially prepared in the exact manner as for the dry test specimen, followed by the addition of test fluid just prior to testing, as follows. First, the thickness and mass of the dry specimen is measured, as described herein, and recorded as initial thickness to the nearest 0.01 mm and initial mass to the nearest 0.001 g. Next, the dry specimen is fully submersed in the test fluid for 60 seconds. After 60 seconds elapse, the specimen is removed from the test fluid and oriented vertically for 30 seconds to allow any excess fluid to drip off. Now the thickness and mass of the wet specimen are measured, as described herein, and recorded as wet specimen caliper to the nearest 0.01 mm and wet specimen mass to the nearest 0.001 g. If desired, the mass of test fluid in the test specimen is calculated by subtracting the initial mass (g) from the wet specimen mass (g) and recording as test specimen fluid amount to the nearest 0.001 g. After the wet test specimen is removed from the test fluid, it must be tested within 10 minutes. In like fashion, five replicate wet test specimens are prepared.

Program the universal test frame for a flexural bend test, to move the crosshead such that the top fixture moves down with respect to the lower fixture at a rate of 1.0 mm/sec until the upper bar touches the top surface of the specimen with a nominal force of 0.02 N, then continue for an additional 12 mm. The crosshead is then immediately returned to the original gage at a rate of 1.0 mm/s. Force (N) and displacement (mm) data are continuously collected at 100 Hz throughout the test.

Load a dry test specimen such that it spans the two lower bars and is centered under the upper bar, with its sides parallel to the bars. For MD bending, the MD direction of the test specimen is perpendicular to the length of the 3 bars. Start the test and continuously collect force and displacement data.

Construct a graph of force (N) versus displacement (mm). From the graph, determine the maximum peak force and record as dry MD peak load to the nearest 0.01 N. Now calculate the maximum slope of the curve between initial force and maximum force (during the loading portion of the curve) and record to the nearest 0.1 unit. Calculate the modulus as follows, and record as dry MD modulus to the nearest 0.001 N/mm².

$$CD or MD Dry or Wet Bending Modulus ( N / {mm}^{2} ) = ( Slope \times ( {Span}^{3} ) ) / ( 4 \times specimen width \times ( specimen {caliper}^{3} ) )$$

Calculate bending stiffness as follows, and record as dry MD bending stiffness to the nearest 0.1 N mm².

$$CD or MD Dry or Wet Bending Stiffness ( N {mm}^{2} ) = Modulus \times Moment of Inertia where Moment of Inertia ( {mm}^{4} ) = ( specimen width \times ( specimen {caliper}^{3} ) ) / 12$$

In like fashion, the procedure is repeated for all five replicates of the dry test specimens. The arithmetic mean among the five replicate dry test specimens is calculated for each of the parameters and reported as Dry Specimen ‘Caliper’ to the nearest 0.01 mm, Dry Specimen Basis Weight to the nearest 0.01 g/m², Dry Specimen Density to the nearest 0.001 g/cm³, Dry CD or MD Peak Load to the nearest 0.01 N, Dry CD or MD Bending Modulus to the nearest 0.001 N/mm², and Dry CD or MD Bending Stiffness to the nearest N mm².

The overall procedure is now repeated for all five replicates of the wet test specimens, reporting results as Wet CD or MD Peak Load to the nearest 0.01 N, Wet CD or MD Bending Modulus to the nearest 0.001 N/mm², and Wet CD or MD Bending Stiffness to the nearest N mm².

### Wet and Dry CD Ultra Sensitive 3 Point Bending Method

The CD (cross-direction) bending properties of a test sample are measured using an ultra sensitive 3 point bend test on a universal constant rate of extension test frame (a suitable instrument is the MTS Alliance using TestSuite Software, as available from MTS Systems Corp., Eden Prairie, MN, or equivalent) equipped with a load cell appropriate for the forces being measured. The test is executed on dry test specimens as well as wet test specimens. The intention of this method is to mimic deformation created in the x-y plane by a wearer of an absorbent article during normal use. All testing is performed in a room controlled at 23° C.±3 C and 50%±2% relative humidity.

The ultra sensitive 3 point bend method is designed to maximize the force signal to noise ratio when testing materials with very low bending forces. The force signal is maximized by using a high sensitivity load cell (e.g., 5N), using a small span (load is proportional to the span cubed) and using a wide specimen width (total measured load is directly proportional to width). The fixture is designed such that the bending measurement is performed in tension, allowing the fixture mass to be kept to a minimum. Noise in the force signal is minimized by holding the load cell stationary to reduce mechanical vibration and inertial effect and by making the mass of the fixture attached to the load cell as low as possible.

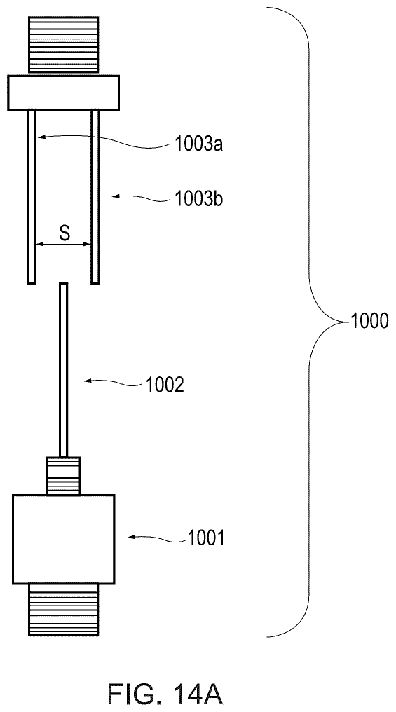

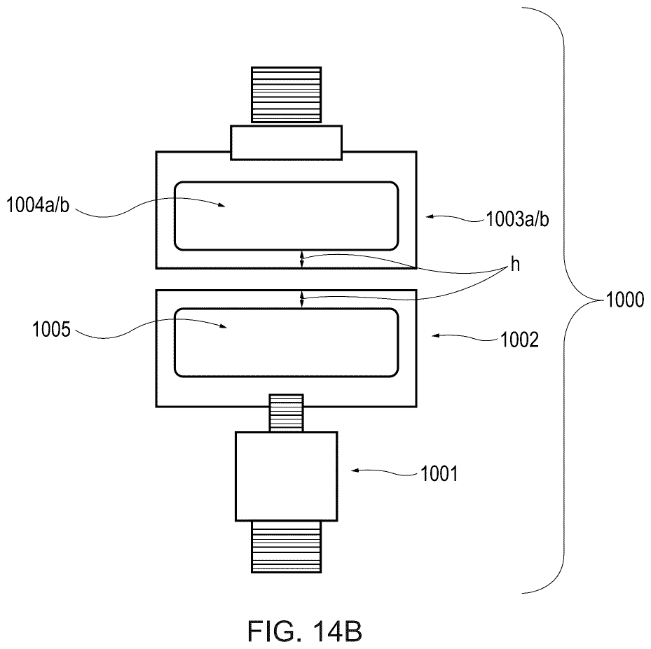

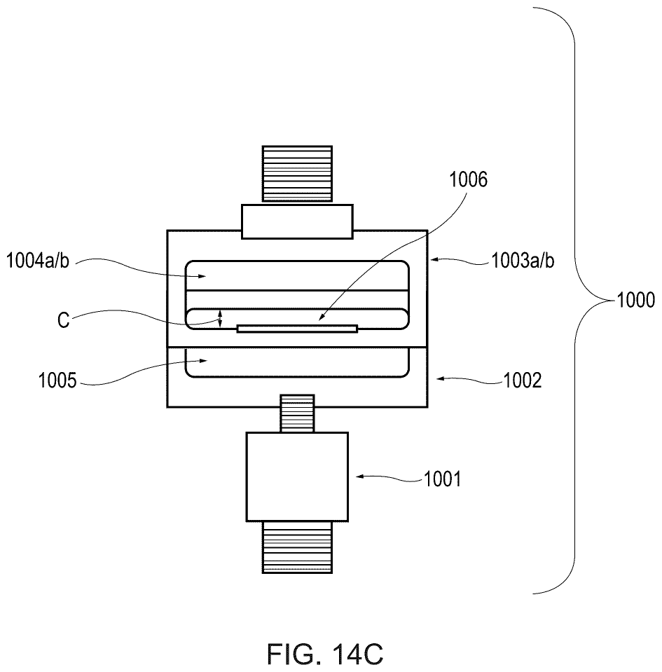

Referring to FIGS. 14A-14C, the load cell 1001 is mounted on the stationary crosshead of the universal test frame. The ultra sensitive fixture 1000 consists of three thin blades constructed of a lightweight, rigid material (such as aluminum, or equivalent). Each blade has a thickness of 1.0 mm, rounded edges and a length that is able to accommodate a bending width of 100 mm. Each of the blades has a cavity 1004a and 1004b (outside blades) and 1005 (central blade) cut out to create a height, h, of 5 mm of blade material along their horizontal edges. The two outside blades 1003a and 1003b are mounted horizontally to the moveable crosshead of the universal test frame, aligned parallel to each other, with their horizontal edges vertically aligned. The span, s, between the two outside blades 1003a and 1003b is 5 mm±0.1 mm (inside edge to inside edge). The central blade 1002 is mounted to the load cell on the stationary crosshead of the universal test frame. When in place, the central blade 1002 is parallel to the two outside blades 1003a and 1003b and centered at the midpoint between the outside blades 1003a and 1003b. The blade fixtures include integral adapters appropriate to fit the respective positions on the universal test frame and lock into position such that the horizontal edges of the blades are orthogonal to the motion of the crossbeam of the universal test frame.

The test fluid used to dose the wet test specimens is prepared by adding 100.0 grams of sodium chloride (reagent grade, any convenient source) to 900 grams of deionized water in a 1-liter Erlenmeyer flask. Agitate until the sodium chloride is completely dissolved.

Samples are conditioned at 23° C.±3 C and 50%±2% relative humidity two hours prior to testing. Dry test specimens are taken from an area of the sample that is free from any seams and residua of folds or wrinkles. The dry specimens are prepared for CD bending (i.e., bending normal to the lateral axis of the sample) by cutting them to a width of 50.0 mm along the CD (cross direction; parallel to the lateral axis of the sample) and a length of 100.0 mm along the MD (machine direction; parallel to the longitudinal axis of the sample), maintaining their orientation after they are cut and marking the body-facing surface (or the surface intended to face the body of a finished article). In like fashion, five replicate dry test specimens are prepared.

Wet test specimens are initially prepared in the exact manner as for the dry test specimen, followed by the addition of test fluid just prior to testing, as follows. The dry specimen is fully submersed in the test fluid for 60 seconds. After 60 seconds elapse, the specimen is removed from the test fluid and oriented vertically for 30 seconds to allow any excess fluid to drip off. After the wet test specimen is removed from the test fluid, it must be tested within 10 minutes. In like fashion, five replicate wet test specimens are prepared.

The universal test frame is programmed such that the moveable crosshead is set to move in a direction opposite of the stationary crosshead at a rate of 1.0 mm/s. Crosshead movement begins with the specimen 1006 lying flat and undeflected on the outer blades 1003a and 1003b, continues with the inner horizontal edge of cavity 1005 in the central blade 1002 coming into contact with the top surface of the specimen 1006, and further continues for an additional 4 mm of crosshead movement. The crosshead stops at 4 mm and then immediately returns to zero at a speed of 1.0 mm/s. Force (N) and displacement (mm) are collected at 50 Hz throughout.

Prior to loading the test specimen 1006, the outside blades 1003a and 1003b are moved towards and then past central blade 1002 until there is approximately a 3 mm clearance, C, between the inner horizontal edges of cavities 1004a and 1004b in the outside blades 1003a and 1003b and the inner horizontal edge of cavity 1005 in the central blade 1002 (see FIG. 14C). The specimen 1006 is placed within clearance C such that it spans the inner horizontal edges of cavities 1004a and 1004b in the outside blades 1003a and 1003b, oriented such that the MD (short side) of the specimen is perpendicular to the horizontal edges of the blades and the body-facing surface of the specimen is facing up. Center the specimen 1006 between the outside blades 1003a and 1003b. Slowly move the outside blades 1003a and 1003b in a direction opposite of the stationary crosshead until the inner horizontal edge of cavity 1005 in the central blade 1002 touches the top surface of the specimen 1006. Start the test and continuously collect force and displacement data.

Force (N) is plotted versus displacement (mm). The maximum peak force is recorded to the nearest 0.001 N. The area under the curve from load onset up to the maximum peak force is calculated and recorded as bending energy to the nearest 0.001 N*mm. The recovery energy is calculated as the area under the curve where the force is unloaded from the maximum peak to 0.0 N and recorded as recovery energy to the nearest 0.001 N*mm. In like fashion, repeat the entire test sequence for a total of five dry test specimens and five wet test specimens.

For each test specimen type (dry and wet), the arithmetic mean of the maximum peak force among like specimens is calculated to the nearest 0.001 N and recorded as Dry Peak Load and Wet Peak load, respectively. For each test specimen type (dry and wet), the arithmetic mean of bending energy among like specimens is calculated to the nearest 0.001 N*mm and reported as Dry Bending Energy and Wet Bending Energy, respectively. For each test specimen type (dry and wet), the arithmetic mean of recovery energy among like specimens is calculated to the nearest 0.001 N*mm and reported as Dry Recovery Energy and Wet Recovery Energy, respectively.

### Wet and Dry Bunched Compression Method

The bunched compression test method measures the force versus displacement behavior across five cycles of load application (“compression”) and load removal (“recovery”) of an absorbent article test sample that has been intentionally “bunched”, using a universal constant rate of extension test frame (a suitable instrument is the MTS Alliance using TestSuite software, as available from MTS Systems Corp., Eden Prairie, MN, or equivalent) equipped with a load cell for which the forces measured are within 1% to 99% of the limit of the cell. The test is executed on dry test specimens as well as wet test specimens that are dosed with a specified amount of test fluid. The intention of this method is to mimic the deformation created in the z-plane of the crotch region of an absorbent article, or components thereof, as it is worn by the wearer during sit-stand movements. All testing is performed in a room controlled at 23° C.±3 C and 50%±2% relative humidity.

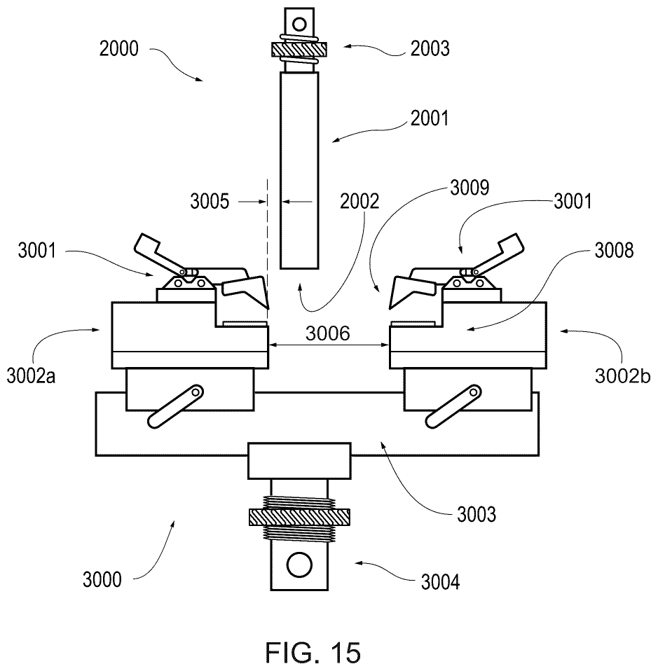

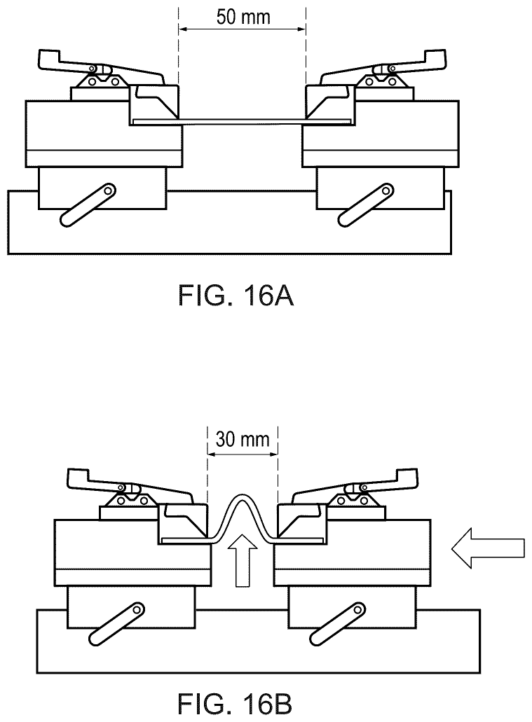

The test apparatus is depicted in FIGS. 15-16B. The bottom stationary fixture 3000 consists of two matching sample clamps 3001 each 100 mm wide, each mounted on its own movable platform 3002a, 3002b. The clamp has a “knife edge” 3009 that is 110 mm long, which clamps against a 1 mm thick hard rubber face 3008. When closed, the clamps are flush with the interior side of its respective platform. The clamps are aligned such that they hold an un-bunched specimen horizontal and orthogonal to the pull axis of the tensile tester. The platforms are mounted on a rail 3003 which allows them to be moved horizontally left to right and locked into position. The rail has an adapter 3004 compatible with the mount of the tensile tester capable of securing the platform horizontally and orthogonal to the pull axis of the tensile tester. The upper fixture 2000 is a cylindrical plunger 2001 having an overall length of 70 mm with a diameter of 25.0 mm. The contact surface 2002 is flat with no curvature. The plunger 2001 has an adapter 2003 compatible with the mount on the load cell capable of securing the plunger orthogonal to the pull axis of the tensile tester.

Test samples are conditioned at 23° C.±3° C. and 50%±2% relative humidity for at least 2 hours before testing. Prepare the test specimen as follows. When testing an intact absorbent article, remove the release paper from any panty fastening adhesive on the garment facing side of the article, if present. Lightly apply talc powder to the adhesive to mitigate any tackiness. If there are cuffs, excise them with scissors so as not to disturb the topsheet or any other underlying layers of the article. Place the article, body facing surface up, on a benchtop. On the article, mark the intersection of the longitudinal midline and the lateral midline. Using a rectangular cutting die or equivalent cutting means, cut a specimen 100 mm in the longitudinal direction by 80 mm in the lateral direction, centered at the intersection of the midlines. When testing a material layer or layered components from an absorbent article, place the material layer or layered components on a benchtop and orient as it would be integrated into a finished article, i.e., identify the body facing surface and the lateral and longitudinal axis. Using a rectangular cutting die, or equivalent cutting means, cut a specimen 100 mm in the longitudinal direction by 80 mm in the lateral direction, centered at the intersection of the midlines. Measure the mass of the specimen and record to the nearest 0.001 grams. Calculate the basis weight of the specimen by dividing the mass (g) by the area (0.008 m²) and record as basis weight to the nearest 1 g/m².

The specimen can be analyzed both wet and dry. The dry specimen requires no further preparation. The test fluid used to dose the wet test specimens is prepared by adding 100.0 grams of sodium chloride (reagent grade, any convenient source) to 900 grams of deionized water in a 1-liter Erlenmeyer flask. Agitate until the sodium chloride is completely dissolved. The wet specimen is dosed with total of 7 ml of the test solution as detailed below.

The liquid dose is added using a calibrated Eppendorf-type pipettor, spreading the fluid over the complete body facing surface of the specimen within a period of approximately 3 sec. The wet specimen is tested 10.0 min±0.1 min after the dose is applied.

Program the tensile tester to zero the load cell, then lower the upper fixture at 2.00 mm/sec until the contact surface of the plunger touches the specimen and 0.02 N is read at the load cell. Zero the crosshead. Program the system to lower the crosshead 15.00 mm at 2.00 mm/sec then immediately raise the crosshead 15.00 mm at 2.00 mm/sec. This cycle is repeated for a total of five cycles, with no delay between cycles. Data is collected at 50 Hz during all compression/decompression cycles.

Position the left platform 3002a 2.5 mm from the side of the upper plunger (distance 3005). Lock the left platform into place. This platform 3002a will remain stationary throughout the experiment. Align the right platform 3002b 50.0 mm from the stationary clamp (distance 3006). Raise the upper probe 2001 such that it will not interfere with loading the specimen. Open both clamps 3001. Referring to FIG. 16A, place the dry specimen with its longitudinal edges (i.e., the 100 mm long edges) within the clamps. With the dry specimen laterally centered, securely fasten both edges in the clamps. Referring to FIG. 16B, move the right platform 3002b toward the stationary platform 3002a a distance of 20 mm so that a separation of 30.0 mm between the left and right clamps is achieved. Allow the dry specimen to bow upward as the movable platform is positioned. Now manually lower the probe 2001 until the bottom surface is approximately 1 cm above the top of the bowed specimen.

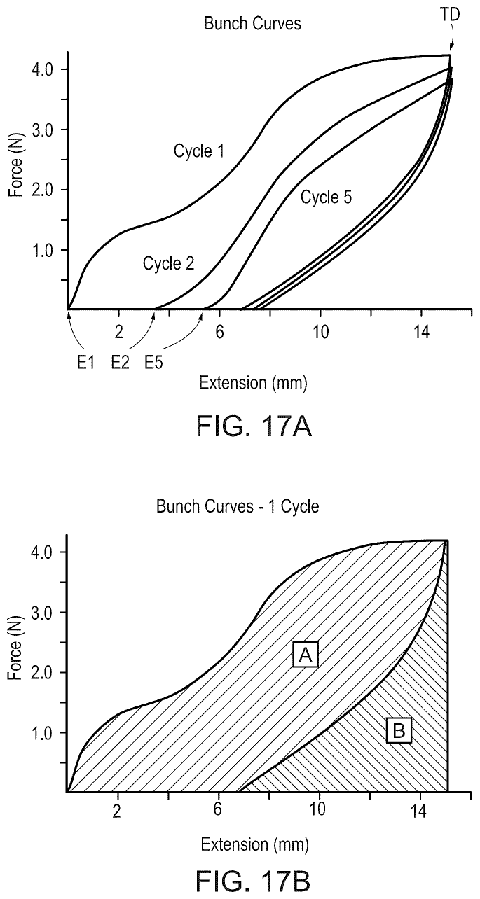

Start the test and continuously collect force (N) versus displacement (mm) data for all five cycles. Construct a graph of force (N) versus displacement (mm) separately for all cycles. A representative curve is shown in FIG. 17A. From the curve, determine the Dry Maximum Compression Force for each Cycle to the nearest 0.01 N, then multiply by 101.97 and record to the nearest 1 gram-force. Calculate the Dry % Recovery between the First and Second cycle as (TD-E2)/(TD−E1)*100 where TD is the total displacement and E2 is the extension on the second compression curve that exceeds 0.02 N, and record to the nearest 0.01%. In like fashion calculate the Dry % Recovery between the First Cycle and other cycles as (TD−E1)/(TD−E1)*100 and record to the nearest 0.01%. Referring to FIG. 17B, calculate the Dry Energy of Compression for Cycle 1 as the area under the compression curve (i.e., area A+B) and record to the nearest 0.1 N*mm. Calculate the Dry Energy Loss from Cycle 1 as the area between the compression and decompression curves (i.e., Area A) and record to the nearest 0.1 N*mm. Calculate the Dry Energy of Recovery for Cycle 1 as the area under the decompression curve (i.e., Area B) and report to the nearest 0.1 N*mm. In like fashion calculate the Dry Energy of Compression (N*mm), Dry Energy Loss (N*mm) and Dry Energy of Recovery (N*mm) for each of the other cycles and record to the nearest 0.1 N-mm. In like fashion, analyze a total of five replicate dry test specimens and report the arithmetic mean among the five dry replicates for each parameter as previously described, including basis weight.

The overall procedure is now repeated for a total of five replicate wet test specimens, reporting results for each of the five cycles as the arithmetic mean among the five wet replicates for Wet Maximum Compression Force to the nearest 1 gram-force for each cycle, Wet Energy of Compression to the nearest 0.1 N*mm for each cycle, Wet Energy Loss to the nearest 0.1 N*mm for each cycle, Wet Energy of Recovery to the nearest 0.1 N*mm for each cycle and Wet % recovery for each cycle. Of particular importance is the 5^{th} cycle wet energy of recovery and 5^{th} cycle wet % recovery properties from this test method.

### CD Cyclic Elongation to 3% Strain Method

The cyclic tensile and recovery response of absorbent article specimens are measured for ten cycles of load application (“elongation”) and load removal (“recovery”) using a universal constant rate of extension test frame. The test specimen is cycled ten times to 3% engineering strain, then back to zero engineering strain. For each cycle, stiffness, peak load, normalized energy to peak, normalized recovery energy, strain at start of cycle, and strain at end of cycle (i.e., “permanent strain”) are calculated and reported. The intention of this method is to understand the ability of samples to stretch in the x-y plane as a result of bodily forces, and then recover to their original state. All measurements are performed in a laboratory maintained at 23° C.±2 C.° and 50%±2% relative humidity and test specimens are conditioned in this environment for at least 2 hours prior to testing.

A suitable universal constant rate of extension test frame is the MTS Alliance interfaced to a computer running TestSuite control software (available from MTS Systems Corp, Eden Prairie, MN), or equivalent. The universal test frame is equipped with a load cell for which forces measured are within 1% to 99% of the limit of the cell. The fixtures used to grip the test specimen are lightweight (<80 grams), vise action clamps with knife or serrated edge grip faces that are at least 40 mm wide. The fixtures are installed on the universal test frame and mounted such that they are horizontally and vertically aligned with one another.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen is cut from an area on the test material that is free of any residual of folds or wrinkles. The test specimen is as long as the lateral length of the article (parallel to the lateral axis of the article, or the intended lateral axis of the article). When excising specimens from absorbent articles of different sizes and widths, the total specimen length (L^{total}) may vary from product to product, thus the results will be normalized to compensate for this variation. The test specimen has a width of 25.4 mm wide (parallel to the longitudinal axis, or intended longitudinal axis of the article). Specimen width (w)=25.4 mm. Measure and record the total specimen length (Lₜₒₜₐₗ) to the nearest 0.1 mm. In like fashion, five replicate test specimens are prepared.

Measure the thickness (t) of the test specimen using a manually-operated micrometer equipped with a pressure foot capable of exerting a steady pressure of 0.1 psi±0.01 psi. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat circular moveable face with a diameter no greater than 25.4 mm. The test specimen is supported by a horizontal flat reference platform that is larger than and parallel to the surface of the pressure foot. Zero the micrometer against the horizontal flat reference platform. Place the test specimen onto the platform, centered beneath the pressure foot. The pressure foot is lowered by hand with a descent rate of 3±1 mm/s until the full weight of the pressure is exerted onto the specimen. After 5 seconds elapse, the thickness is recorded as specimen thickness (t) to the nearest 0.01 mm.

Prepare the universal test frame as follows. Set the initial grip to grip separation distance to a nominal gage length (Lₙₒₘᵢₙₐₗ) that is shorter than the total specimen length and such that the specimen can be gripped securely at both ends (i.e., Lₙₒₘᵢₙₐₗ<Lₜₒₜₐₗ). Then zero the crosshead. Program the test frame to move the grips closer together by an intentional slack of 1 mm to ensure no pretension force exists on the test specimen at the onset of the test. (During this motion, the specimen will become slack between the tensile grips.) Next, the grips will move apart at a slack speed of 1 mm/s until the slack preload of 0.05 N is exceeded. At this point, the following are true. 1) The crosshead position signal (mm) is defined as the specimen slack (L_{slack}). 2) The initial specimen gage length (L₀) is calculated as the nominal gage length plus the slack L₀=Lₙₒₘᵢₙₐₗ+L_{slack}, where units are in millimeters. 3) The crosshead extension (ΔL) is set to zero (0.0 mm). 4) The crosshead displacement (mm) is set to zero (0.0 mm). At this position the engineering strain is zero, 0.0. Engineering strain is calculated as the change in length (ΔL) divided by the initial length (L₀). Engineering strain=ΔL/L₀. For one test cycle, the grips move apart at the initial speed of 1 mm/s until the engineering strain endpoint of 0.03 mm/mm is exceeded, immediately followed by the grips moving toward each other at the initial speed of 1 mm/s until the crosshead signal becomes less than the crosshead return position of 0 mm. The test cycle is repeated until a total of 10 cycles is complete.

The test is executed by inserting the test specimen into the grips such that the long axis of the specimen is parallel and centered with the motion of the crosshead. Start the test and continuously collect time, force and displacement data at a data acquisition rate of 100 Hz.

Construct a graph of load (N) versus displacement for all ten cycles. For each cycle, perform the following. Record peak load to the nearest 0.01 N. Calculate the energy to peak (Eₚₑₐₖ) as the area under the load versus displacement curve from the cycle start to the strain endpoint of 0.03 mm/mm (during the loading portion of the cycle) and record to the nearest 0.01 N*mm. Calculate the return energy (Eᵣₑₜᵤᵣₙ) as the area under the load versus displacement curve from the strain endpoint of 0.03 mm/mm to the crosshead return of 0 mm (during the unloading portion of the cycle) and record as recovery energy to the nearest 0.01 N*mm. Calculate the normalized energy to peak (NEₚₑₐₖ) as the energy to peak divided by the initial length, where NEₚₑₐₖ=Eₚₑₐₖ/L₀, and record to the nearest 0.01 mN. Calculate the normalized return energy (NEᵣₑₜᵤᵣₙ) as the return energy divided by the initial length (NEᵣₑₜᵤᵣₙ=Eᵣₑₜᵤᵣₙ/L₀), and record to the nearest 0.01 mN. Units of NEₚₑₐₖ and NEᵣₑₜᵤᵣₙ are milliNewtons (mN).

Now construct a graph of engineering stress (a) versus engineering strain for all ten cycles, and for each cycle perform the following. Engineering stress, in units of N/mm², is the load divided by the cross sectional area of the specimen, where the cross sectional area is the specimen width (w) multiplied by the thickness (t), σ=Load/(w*t). Determine the modulus, or slope of the stress versus strain curve for a line between the point that occurs at the minimum force and the point that occurs at the maximum force (during the loading portion of the cycle) and record as modulus to the nearest 0.01 N/mm. Calculate stiffness by multiplying the modulus by the specimen thickness and record as tensile stiffness to the nearest 0.01 N/mm. The strain of the test specimen at the beginning of the cycle is defined by the strain when the slack preload of 0.05 N is exceeded for that cycle (during the loading portion of the cycle), and is recorded as cycle initial strain to the nearest 0.01 mm/mm. The strain of the test specimen at the end of the cycle is defined by the strain when the load becomes less than the preload of 0.05 N for that cycle (during the unloading portion of the cycle), and is recorded as permanent strain to the nearest 0.01 mm/mm. In like fashion, the overall procedure is now repeated for all five replicates.

The arithmetic mean among the five replicate test specimens is calculated for each of the parameters, for each of the ten cycles, and reported as Peak Load to the nearest 0.01 N, Normalized Energy to Peak to the nearest 0.01 mN, Normalized Recovery Energy to the nearest 0.01 mN, Tensile Stiffness to the nearest 0.01 N/mm, Cycle Initial Strain to the nearest 0.01 mm/mm, and Permanent Strain to the nearest 0.01 mm/mm.

### Nonwoven Thickness—Pressure Method

The thickness of a test specimen is measured as the distance between a reference platform on which the specimen rests and a pressure foot that exerts a specified amount of pressure onto the specimen over a specified amount of time. For purposes herein, thickness is measured and reported at two different confining pressures (7 g/cm² and 70 g/cm²). All measurements are performed in a laboratory maintained at 23° C.±2 C.° and 50%±2% relative humidity and test specimens are conditioned in this environment for at least 2 hours prior to testing.

Thickness is measured with a manually-operated micrometer equipped with a pressure foot capable of exerting a steady pressure (7 g/cm² and 70 g/cm²) onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm; however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Measurements are made on test specimens taken from rolls or sheets of the raw material, or test specimens obtained from a material layer removed from an absorbent article. When excising the material layer from an absorbent article, use care to not impart any contamination or distortion to the layer during the process. The excised layer should be free from residual adhesive and any fibers that may have transferred from underlying layers. To ensure that all adhesive and any transferred fibers are removed, soak the layer in a suitable solvent that will dissolve the adhesive and release any transferred fibers present without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained from an area free of folds or wrinkles, and it must be larger than the pressure foot.

To measure thickness at a confining pressure of 7 g/cm², first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the thickness of the test specimen to the nearest 0.01 mm. In like fashion, repeat for a total of ten replicate test specimens. Calculate the arithmetic mean for all thickness measurements obtained at a confining pressure of 7 g/cm² and report as Thickness at 7 g/cm² to the nearest 0.01 mm.

To measure thickness at a confining pressure of 70 g/cm², first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the thickness of the test specimen to the nearest 0.01 mm. In like fashion, repeat for a total of ten replicate test specimens. Calculate the arithmetic mean for all thickness measurements obtained at a confining pressure of 70 g/cm² and report as Thickness at 70 g/cm² to the nearest 0.01 mm.

### Acquisition Time and Rewet Method

This method describes how to measure gush acquisition time, interfacial free fluid amount as well as low and high pressure rewet values for an absorbent article loaded with new Artificial Menstrual Fluid (nAMF; preparation provided separately herein). A pretreatment step is followed by three introductions of known volumes of nAMF to the absorbent article. The time required for the absorbent article to acquire each of the doses of nAMF is measured using a strikethrough plate and an electronic circuit interval timer. After each liquid dose, Interfacial Free Fluid (IFF) is measured gravimetrically as fluid is transferred from the bottom surface of the strikethrough plate to filter paper. Subsequently, low and high pressure rewet are measured after the last liquid dose. Surface Free Fluid (SFF) is the amount of fluid that remains in the topsheet of the absorbent article. SFF is measured by performing low pressure (0.1 psi) rewet. Immediately after measuring SFF, a higher pressure (0.5 psi) rewet is performed to determine the overall rewet of the absorbent article. All testing is performed in a room maintained at 23° C.±2 C.° and 50%±2% relative humidity.

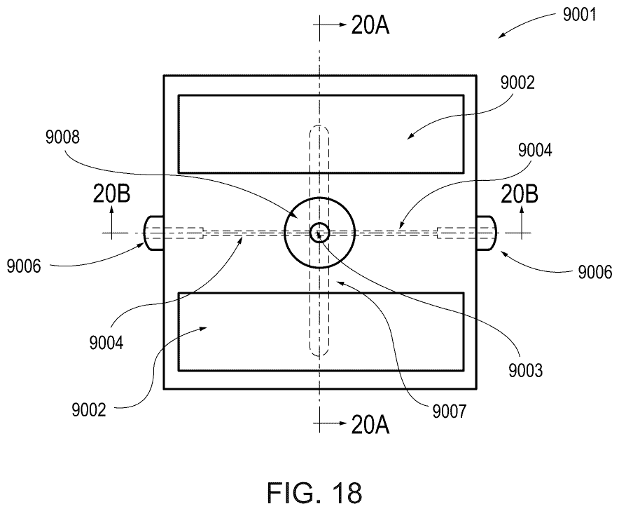

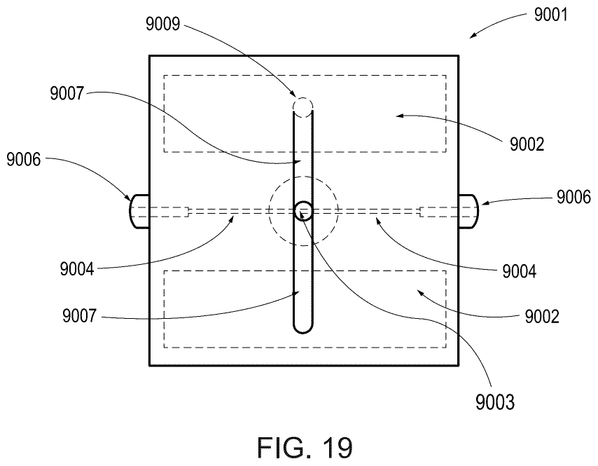

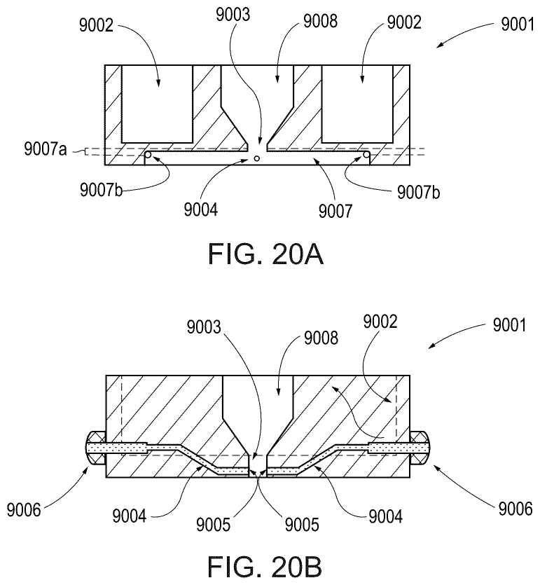

Referring to FIGS. 18-20B, the strikethrough plate 9001 is constructed of Plexiglas, or equivalent, with an overall dimension of 10.2 cm long by 10.2 cm wide by 3.1 cm tall. A central, test fluid well 9008 has a circular opening of 25 mm in diameter is located at the top plane of the plate with initial lateral walls that extend 15 mm deep at a 90° angle and then slope downward at an angle of 82° for an additional depth of 7.5 mm to reach the test fluid reservoir 9003. The test fluid reservoir 9003 is concentric to the test fluid well 9008 and has a diameter of 6.6 mm with lateral walls that extend 5 mm deep at a 90° angle. The test fluid reservoir 9003 opens into the longitudinal fluid channel 9007 located at the bottom of the plate. The longitudinal fluid channel 9007 has lateral walls that initially extend 3.5 mm deep at the midpoint of the channel (just beneath the test fluid reservoir 9003), then slant downward at an angle 9007a of 0.72° towards each longitudinal end of the channel to a final depth of 3 mm. The longitudinal fluid channel opens to the bottom plane of the plate for the fluid to be introduced onto the underlying test sample. The longitudinal fluid channel 9007 is centered over the test fluid reservoir 9003 and extends in a direction that is perpendicular to the electrodes 9004. The longitudinal fluid channel 9007 has a width of 5 mm and a length of 80 mm, with lateral edges that are rounded with a radius 9007b of 1.0 mm. The longitudinal ends of the longitudinal fluid channel 9007 are rounded with a radius 9009 of 2.5 mm. Two wells 9002 (80.5 mm long by 24.5 mm wide by 25 mm deep) located outboard of the fluid reservoir, are filled with stainless steel shot (or equivalent) to adjust the overall mass of the plate to provide a constraining pressure of 0.10 psi (7.0 g/cm²) to the Test Area. The procedure for determining the test area is subsequently described herein. Electrodes 9004 are embedded in the plate 9001, connecting the exterior banana jacks 9006 to the inside wall 9005 of the longitudinal fluid channel 9003. A circuit interval timer is plugged into the jacks 9006, monitors the impedance between the two electrodes 9004, and measures the time from introduction of the nAMF into reservoir 9003 until the nAMF drains from the reservoir. The timer has a resolution of 0.01 sec.

A pretreatment plate is used in combination with a pretreatment weight to apply tiny droplets of nAMF to the surface of the test sample as a means to prime the surface of the test sample prior to the introduction of the full liquid dose. The pretreatment plate is constructed of Plexiglass, or equivalent, that is 14 inch (35.6 cm) long by 8 inch (20.3 cm) wide with a thickness of about 0.25 inch (6.4 mm). The pretreatment plate has five circular markers, each 5 mm in diameter, placed 1 cm apart (center to center) that are aligned along the longitudinal axis of the plate. The central marker is centered at the lateral midpoint of the plate. These markers indicate the placement of the nAMF droplets. The markers are located on the underside of the pretreatment plate and can be milled out or simply drawn on with a permanent marker, or equivalent. The pretreatment weight is 10.2 cm×10.2 cm and consists of a flat, smooth rigid material (e.g. stainless steel) with an optional handle. The pretreatment weight (including optional handle) has a total mass of 726 g±0.5 g to give a pressure of 0.10 psi (7.0 g/cm²) across the bottom surface area of the weight.

When measuring the interfacial fluid amounts, a rubber pad is used to provide a reproducibly flat surface that enables even pressure distribution. The IFF rubber pad is constructed from high strength neoprene rubber with 40 A durometer and a thickness of ⅛ inch (available from W.W. Grainger, Inc, item #1DUV4, or equivalent) and cut to dimensions of 6 inch (15.2 cm) by 6 inch (15.2 cm).

For the overall rewet portion of the test, a padded weight assembly that applies 0.5 psi (35.1 g/cm²) to the Test Area is required. The procedure for determining the test area is subsequently described herein. The rewet weight is constructed as follows. Lay a piece of polyethylene film (about 25 microns thick, any convenient source) horizontally flat on a rigid bench surface. A piece of polyurethane foam (25 mm thick, density of 1.0 lb/ft³, IDL 24 psi, available from Concord-Renn Co. Cincinnati, OH, or equivalent) is cut to 10.2 cm by 10.2 cm and then laid centered on top of the film. A piece of Plexiglas (10.2 cm by 10.2 cm and about 6.4 mm thick) is then stacked on top of the polyurethane foam. Next the polyethylene film is used to wrap the polyurethane foam and Plexiglas plate securing it with transparent tape. A metal weight with handle is stacked on top of, and fastened to, the Plexiglass plate such that the total mass of the padded weight assembly can be adjusted to apply a pressure of 0.5 psi (35.1 g/cm²) to the Test Area.

For the IFF, SFF and overall rewet steps, various layers of filter paper are required. The filter paper is conditioned at 23° C.±2 C.° and 50%±2% relative humidity for at least 2 hours prior to testing. A suitable filter paper has a basis weight of about 88 gsm, a thickness of about 249 microns with an absorption rate of about 5 seconds, and is available from Ahlstrom-Munksjo (Mt. Holly Springs, PA) as grade 632, or equivalent. The filter paper has dimensions of 5 inch by 5 inch (12.7 cm by 12.7 cm).

Test samples are conditioned at 23° C.±2 C.° and 50%±2% relative humidity for at least 2 hours prior to testing. Test samples are removed from their outer packaging and the wrappers are opened to unfold the product, if applicable, using care not to press down or pull on the products while handling. No attempt is made to smooth out wrinkles. Tear the release paper between the wings, if applicable, and lay the sample on a horizontally flat, rigid surface with the body-side facing up (e.g. panty-side down). Determine the dose location as follows. For symmetrical products (i.e. the front of the product is the same shape and size as the back of the product when laterally divided along the midpoint of the longitudinal axis of the product), the dose location is the intersection of the midpoints of the longitudinal and lateral axes of the absorbent core. For asymmetrical products (i.e. the front of the product is not the same shape and size as the back of the product when laterally divided along the midpoint of the longitudinal axis of the product), the dose location is the midpoint of the product's wings at the lateral midpoint of the absorbent core. For products that have a foam core with holes and slits either punched out or printed, the dose location is the longitudinal midpoint of the hole-punched (or hole-printed) region at the lateral midpoint of the absorbent core. Once determined, mark the dose location with a small dot using a black, fine-tip, permanent marker. If wings are present, fold them to the back of the product.

Determine the Test Area of the test sample, as follows. This area will be used so that the mass of the strikethrough plate and the mass of the rewet weight can be properly adjusted to deliver the required pressure (0.1 psi and 0.5 psi, respectively). Measure the width of the absorbent core of the test sample as the distance between one lateral edge of the core to the other lateral edge of the core along a line that is positioned at the dosing location and runs perpendicular to the longitudinal axis of the test sample, and record as core width to the nearest 0.01 cm. Now multiply the core width by 10.2 cm (the length of the strikethrough plate and rewet weight) and record as Test Area to the nearest 0.1 cm². The total mass of the strikethrough plate is the Test Area multiplied by 7 g/cm². The total mass of the rewet weight is the Test Area multiplied by 35.1 g/cm².

The test sample is pretreated with nAMF as follows. Place the pretreatment plate onto a horizontally flat, rigid surface such that the side with the circular markers is facing down. Using a single channel, fixed volume pipettor, accurately dispense 50 μL of nAMF onto the topside of the pretreatment plate at the location of each of the five circular markers. Position the test sample above the pretreatment plate such that the body-side of the sample is facing the plate, the longitudinal axis of the sample and plate are aligned, and the pre-marked dose location on the test sample is centered over the central droplet of nAMF on the pretreatment plate. After properly positioned, place the test sample into contact with the pretreatment plate, then immediately apply the pretreatment weight onto the back side of the test sample, centering it over the dose location/central droplet of nAMF on the pretreatment plate. Start a 40 second timer. After 40 seconds have elapsed, remove the pretreatment weight from the test sample and remove the test sample from the pretreatment plate. Invert the test sample so that the body-side is facing up, place it onto a horizontally flat, rigid surface and immediately proceed with the steps that follow.

The first acquisition time (ACQ-1) is measured as follows. Connect the electronic circuit interval timer to the strikethrough plate 9001 and zero the timer. Position the strikethrough plate 9001 above the body-side of the test sample such that the long axis of the longitudinal fluid channel 9007 on the underside of the strikethrough plate 9001 is aligned with the longitudinal axis of the test sample, and ensure that the fluid reservoir 9003 is centered over the pre-marked dose location on the test sample. To note, nAMF should be visible through the fluid reservoir 9003 at the dose location on the test sample. After properly positioned, gently place the strikethrough plate 9001 onto the test sample. Using an adjustable volume pipettor, accurately dispense 2.0 mL of nAMF into the fluid well 9008 in the strikethrough plate 9001. The fluid is dispensed, without splashing, along the angled walls of the fluid well 9008 within a period of 3 seconds or less. Immediately after the fluid has been acquired, record the first acquisition time (ACQ-1) displayed on the circuit interval timer to the nearest 0.1 seconds. Leave the strikethrough plate 9001 in place on the test sample, and immediately start a 2 minute timer.

After 2 minutes have elapsed, measure the first Interfacial Free Fluid (IFF-1) as follows. Place the IFF rubber pad onto a horizontally flat, rigid surface. Measure the mass of one layer of filter paper to the nearest 0.0001 g and record as IFF-1ᵢₙᵢₜᵢₐₗ. Place the filter paper centered onto the IFF rubber pad. Transfer the strikethrough plate 9001 from the test sample to the pre-weighed filter paper such that the plate is centered on the filter paper, and immediately start an 8 minute timer. After 10 seconds have elapsed on the 8 minute timer, remove the strikethrough plate from the filter paper and gently replace it back onto the test sample, exactly as previously positioned. Within the next 10 seconds, measure the mass of the filter paper to the nearest 0.0001 g and record as IFF-1_{final}.

The second acquisition time (ACQ-2) is measured as follows. After 8 minutes have elapsed, apply the second gush of fluid using an adjustable volume pipettor to accurately dispense 4.0 mL of nAMF into the fluid well 9008 in the strikethrough plate 9001, as previously described. Immediately after the fluid has been acquired, record the second acquisition time (ACQ-2) displayed on the circuit interval timer to the nearest 0.1 second. Leave the strikethrough plate 9001 in place on the test sample, and immediately start a 2 minute timer.

After 2 minutes have elapsed, measure the second Interfacial Free Fluid (IFF-2) as follows. Place the IFF rubber pad onto a horizontally flat, rigid surface. Measure the mass of a fresh, single layer of filter paper to the nearest 0.0001 g and record as IFF-2ᵢₙᵢₜᵢₐₗ. Place the filter paper centered onto the IFF rubber pad. Transfer the strikethrough plate 9001 from the test sample to the pre-weighed filter paper such that the plate is centered on the filter paper and immediately start an 8 minute timer. After 10 seconds have elapsed on the 8 minute timer, remove the strikethrough plate 9001 from the filter paper and gently replace it back onto the test sample, exactly as previously positioned. Within the next 10 seconds, measure the mass of the filter paper to the nearest 0.0001 g and record as IFF-2_{final}.

The third acquisition time (ACQ-3) is measured as follows. After 8 minutes have elapsed, apply the third gush of fluid using an adjustable volume pipettor to accurately dispense 2.0 mL of nAMF into the fluid well 9008 in the strikethrough plate 9001, as previously described. Immediately after the fluid has been acquired, record the third acquisition time (ACQ-3) displayed on the circuit interval timer to the nearest 0.1 second. Leave the strikethrough plate 9001 in place on the test sample, and immediately start a 2 minute timer.

After 2 minutes have elapsed, measure the third Interfacial Free Fluid (IFF-3) as follows. Place the IFF rubber pad onto a horizontally flat, rigid surface. Measure the mass of a fresh, single layer of filter paper to the nearest 0.0001 g and record as IFF-3ᵢₙᵢₜᵢₐₗ. Place the filter paper centered onto the IFF rubber pad. Transfer the strikethrough plate 9001 from the test sample to the pre-weighed filter paper such that the plate is centered on the filter paper and immediately start an 8 minute timer. After 10 seconds have elapsed on the 8 minute timer, remove the strikethrough plate 9001 from the filter paper and set it on its side so that the pad-side of the plate is not contacting the bench. Within the next 10 seconds, measure the mass of the filter paper to the nearest 0.0001 g and record as IFF-3_{final}.

Measure Surface Free Fluid (SFF) as follows. After 8 minutes have elapsed, measure the mass of a fresh stack of 5 filter papers to the nearest 0.0001 g and record as SFFᵢₙᵢₜᵢₐₗ. Place the stack of filter papers on top of the body-side of the test sample such that they are centered over the dose location. Now gently place the strikethrough plate 9001 on top of the filter papers such that the pad-side of the plate is centered on the filter papers, and immediately start a 10 second timer. After 10 seconds have elapsed, remove the strikethrough plate 9001 from the filter papers and set it aside. Measure the mass of the stack of 5 filter papers to the nearest 0.0001 g and record as SFF_{final}. Immediately proceed to the next step.

Measure overall rewet as follows. Measure the mass of a fresh stack of 5 filter papers to the nearest 0.0001 g and record as REWETᵢₙᵢₜᵢₐₗ. Place the filter papers on top of the body-side of the test sample such that they are centered over the dose location. Now place the padded Rewet Weight on top of the stack of filter papers such that the weight is centered on the filter paper stack, and immediately start a 30 second timer. After 30 seconds have elapsed, remove the rewet weight and measure the mass of the stack of 5 filter papers to the nearest 0.0001 g, then record as REWET_{final}. Discard the sample and thoroughly clean and then dry the fluid well 9008, fluid reservoir 9003, longitudinal fluid channel 9007 and the bottom surface of the strikethrough plate 9001 prior to testing the next sample.

Make the following calculations for each of the parameters measured, as follows. Calculate Total Gush Absorbency Time as the sum of ACQ-1, ACQ-2 and ACQ-3, and record to the nearest 0.1 second. Calculate IFF-1 by subtracting IFF-1ᵢₙᵢₜᵢₐₗ from IFF-1_{final}, and record to the nearest 0.0001 g. Calculate IFF-2 by subtracting IFF-2ᵢₙᵢₜᵢₐₗ from IFF-2_{final}, and record to the nearest 0.0001 g. Calculate IFF-3 by subtracting IFF-3ᵢₙᵢₜᵢₐₗ from IFF-3_{final}, and record to the nearest 0.0001 g. Calculate Total IFF as the sum of IFF-1, IFF-2 and IFF-3, and record to the nearest 0.1 g. Calculate SFF by subtracting SFFᵢₙᵢₜᵢₐₗ from SFF_{final}, and record to the nearest 0.0001 g. Calculate Total IFF+SFF as the sum of Total IFF and SFF, and record to the nearest 0.1 g. Calculate Overall Rewet by subtracting REWETᵢₙᵢₜᵢₐₗ from REWET_{final}, and record to the nearest 0.0001 g.

The entire procedure is repeated for a total of three replicate test samples. The reported value for each of the parameters is the arithmetic mean of the three individually recorded measurements for each Acquisition Time (ACQ-1, ACQ-2 and ACQ-3) to the nearest 0.1 seconds, Total Gush Absorbency Time to the nearest 0.1 seconds, Interfacial Free Fluid (IFF-1, IFF-2 and IFF-3) to the nearest 0.0001 g, Total IFF to the nearest 0.1 g, Surface Free Fluid (SFF) to the nearest 0.0001 g, Total IFF+SFF to the nearest 0.1 g, and Overall Rewet to the nearest 0.0001 g.

New Artificial Menstrual Fluid (nAMF) Preparation

This formulation of new Artificial Menstrual Fluid (nAMF) is composed of a mixture of defibrinated sheep blood, a phosphate buffered saline solution and a mucous component. The nAMF is prepared such that it has a viscosity between 7.40 to 9.00 centipoise at 23° C.

Viscosity of the nAMF is performed using a low viscosity rotary viscometer (a suitable instrument is the Brookfield DV2T fitted with a Brookfield UL adapter, available from AMETEK Brookfield, Middleboro, MA, or equivalent). The appropriate size spindle for the viscosity range is selected, and the instrument is operated and calibrated as per the manufacturer. Measurements are taken at 23° C.±1 C.° and at 60 rpm. Results are reported to the nearest 0.01 centipoise.

Reagents needed for the nAMF preparation include: defibrinated sheep blood with a packed cell volume of 38% or greater (collected under sterile conditions, available from Cleveland Scientific, Inc., Bath, OH, or equivalent), gastric mucin with a viscosity target of 3-4 centistokes when prepared as a 2% aqueous solution (crude form, sterilized, available from American Laboratories, Inc., Omaha, NE, or equivalent), sodium phosphate dibasic anhydrous (reagent grade), sodium chloride (reagent grade), sodium phosphate monobasic monohydrate (reagent grade), sodium benzoate (reagent grade), benzyl alcohol (reagent grade) and distilled water, each available from VWR International or equivalent source.

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add distilled water to volume. Mix thoroughly. To prepare about 200 mL of phosphate buffered saline solution, add 49.50 g±0.10 g of Solution A and 157.50 g±0.10 g of Solution B to a sufficiently size bottle that has a lid with a good seal. Then add 1.0 g of sodium benzoate and 1.60 g of benzyl alcohol to the bottle along with a stir bar and set aside.

The mucous component of the nAMF is a mixture of the phosphate buffered saline solution and gastric mucin. The amount of gastric mucin added to the mucous component directly affects the final viscosity of the prepared nAMF. To determine the amount of gastric mucin needed to achieve nAMF within the target viscosity range (7.4-9.0 centipoise at 23° C. and 60 rpm), prepare 3 batches of nAMF with varying amounts of gastric mucin in the mucous component, and then interpolate the exact amount needed from a concentration versus viscosity curve with a least squares linear fit through the three points. A successful range of gastric mucin is usually between 13 to 15 grams per 400 mL batch of nAMF, although this can vary significantly based upon the supplier, age, and lot of mucin.

To prepare about 200 mL of the mucous component, add the pre-determined amount of gastric mucin to the bottle containing the previously prepared phosphate buffered solution and then apply the lid. Place the bottle on a wrist-action shaker for 5 minutes at the highest speed. After 5 minutes, remove the flask of mucous component from the wrist-action shaker and place onto a magnetic stir plate. Stir for at least 2 hours until there are no lumps of mucin present, then remove the stir bar from the flask. Using a homogenizer, blend the mucous component for 5 minutes at 10,000 rpm. A suitable homogenizer is the T18 Ultra-Turrax fitted with a S18N-19G dispersing tool (19 mm stator diameter, 12.7 mm rotor diameter, 0.4 mm gap between rotor and stator), both available from IKA Works, Inc, Wilmington, NC, or equivalent. After the final mixing step, measure and record the viscosity of the mucous component to the nearest 0.01 centipoise at 23° C.±1 C.° and at 20 rpm using the viscometer with the UL adapter. Ensure that the viscosity of the prepared mucous component is within the target range of 9.0-11.0 centipoise.

The nAMF is a 50:50 mixture of the mucous component and sheep blood. Ensure the temperature of the sheep blood and mucous component are 23° C.±1 C.°. To prepare about 400 mL of nAMF, add 200 g of the mucous component to a glass bottle with at least 500 mL capacity. Now add 200 g of sheep blood to the bottle along with a stir bar. Mix on a magnetic stir plate until thoroughly combined. Ensure the viscosity of the prepared nAMF is within the target range of 7.4-9.0 centipoise when measured at 23° C.±1 C.° and 60 rpm using the viscometer with the UL adapter. If the viscosity is too high, it can be adjusted by adding the previously prepared phosphate buffered saline solution in 0.5 g increments followed by stirring for 2 minutes and then re-checking the viscosity until the target range is reached.

The qualified nAMF should be refrigerated at 4° C. unless intended for immediate use. nAMF may be stored in an air-tight container at 4° C. for up to 48 hours after preparation. Prior to testing, the nAMF must be brought to 23° C.±1 C.°. Any unused portion is discarded after testing is complete.

### In-Bag Compression Recovery Method

In the product development of lofty cellulose rich absorbent articles, it is important to understand the caliper recovery of said articles after they are compressed and held in a package for an extended time period. The In-Bag Compression Recovery method measures the caliper recovery of a stack of absorbent articles taken from a sealed full bag package where they have been subjected to long-term compressive forces. Caliper recovery is calculated by comparing the initial stack height inside the package to the stack height measured at specified timepoints after the stack of absorbent articles have been removed from the package. All testing is performed in a room controlled at 23° C.±3 C.° and 50%±2% relative humidity.

Package dimensions and stack height measurements are made using a Universal Package Tester (UPT) equipped with a motorized test stand that includes a control box with a touch screen. A suitable instrument is available from Alluris GmbH & Co. (Freiburg, Germany) as model FMT-310, or equivalent. The stationary base plate of the UPT has a surface area larger than the surface area of the stack of test samples. A compression plate that has a diameter of 150 mm is mounted to the load cell (5 N) that is attached to the moveable crossbeam of the test stand. The position of the compression plate is zeroed and a reference (i.e., “home”) position is set according to the manufacturer's instructions such that accurate distances can be measured between the contact surface of the base plate and the contact surface of the compression plate. Ensure that the compression plate is secured such that its contact surface face is parallel to the base plate and oriented orthogonal to the motion of the crossbeam of the test stand. The system is calibrated per the manufacturer's instructions prior to testing.

Test samples are prepared as follows. Obtain a full bag test package of absorbent article pads with a production date that is at least 1 week prior to the date of testing. Referring to FIGS. 12A-13 as guidance, determine the direction of the compression stack axis 510 of the full bag test package 500, where the compression stack axis is defined as the imaginary line that begins at the flat side of the first folded absorbent article pad in the stack and extends through the orderly pile of folded pads to end at the flat side of the last folded absorbent article pad in the stack contained inside the package. The compression stack axis is orthogonal to the flat sides of the folded absorbent article pads and represents the direction that the filled bag width (FBW) 508 of the package will be measured, as described herein. Mark the direction of the compression stack axis on the outside of the package using a permanent ink pen. In like fashion, prepare a total of three replicate full bag test packages. The full bag test packages are conditioned in a room maintained at 23° C.±3 C.° and 50%±2% relative humidity for at least 2 hours prior to testing.

Execute the package dimensions and stack height measurement of the full bag test package using the UPT as follows. Place the test package onto the base plate of the UPT such that the pre-identified direction of the compression stack axis is parallel to the motion of the crossbeam of the test stand and orthogonal to the contact surface of the base plate. Position the test package such that a single stack of folded pads within the package is centered beneath the compression plate. Set a start position on the UPT such that the distance between the compression plate and the base plate is about 1 cm greater than the approximate filled bag width (FBW) of the package. Program the UPT as follows. Lower the compression plate from the home position to the pre-set start position at a rate of 180 mm/min. The compression plate is then further lowered at a rate of 12 mm/min until it makes contact with the test package, then continues to lower until a force of 1 N (±0.05 N) is reached, stops briefly, and then immediately moves back to the pre-set start position. The height of the compression plate at which the 1 N force was reached is the filled bag width (FBW) and is recorded as In-Bag Stack Height to the nearest 0.1 mm. Now position the test package onto the base plate of the UPT such that the height of the package can be measured. To make the package height measurement, position the package such that the pre-identified direction of the compression stack axis is orthogonal to the motion of the crossbeam of the test stand and parallel to the contact surface of the base plate. Center the test package beneath the compression plate. Set a start position on the UPT such that the distance between the compression plate and the base plate is about 1 cm greater than the approximate filled bag height of the package. Measure the filled bag height with the UPT in the same manner as described for the FBW measurement, where the compression plate moves from the pre-set start position toward the full bag package at a rate of 12 mm/min until a force of 1 N force is applied to the package and then the plate immediately moves back to the pre-set start position. The height of the compression plate at which the 1 N force was reached is recorded as filled bag height (FBH) the nearest 0.1 mm. Now position the test package onto the base plate of the UPT such that the depth of the package can be measured. To make the package depth measurement, position the package such that the side of the package where the direction of the compression stack axis was marked is oriented such that it faces the base plate of the UPT. Center the test package beneath the compression plate. Set a start position on the UPT such that the distance between the compression plate and the base plate is about 1 cm greater than the approximate filled bag depth of the package. Measure the filled bag depth with the UPT in the same manner as described for the FBW measurement, where the compression plate moves from the pre-set start position toward the full bag package at a rate of 12 mm/min until a force of 1 N force is applied to the package and then the plate immediately moves back to the pre-set start position. The height of the compression plate at which the 1 N force was reached is recorded as filled bag depth (FBD) the nearest 0.1 mm. Now measure the mass of the full bag test package and record as filled bag mass (FBM) to the nearest 0.01 g.

Execute the stack height measurement of the stack of pads after they are removed from the test package as follows. Carefully open the full bag test package such that no pressure is exerted onto the package or the folded pads within. Now remove the pads from the package, stacking them in such a way as to mimic how they were stacked inside the package, and start a 2 minute timer. Set the empty package aside for now. Without undue delay, count the number of folded pads that make up the single stack for which the FBW was just measured and record this value as # of Pads Per Stack in Bag. Also count the number of individual stacks of pads that are present in the full bag package and record this value as Stacks Per Bag (SPB). Without unwrapping or unfolding the pads, transfer the stack of pads to the base plate of the UPT. Ensure that the stack is properly aligned with the individual pads lying one on top of another and ensure that the number of pads in the stack is equal to the # of Pads Per Stack in Bag. Orient the stack in the same manner as described for the FBW measurement, with the compression stack axis parallel to the motion of the crossbeam of the test stand and orthogonal to the contact surface of the compression plate. Position the stack such that it is centered beneath but not contacting the compression plate and set a start position on the UPT such that the distance between the compression plate and the base plate is about 1 cm greater than the approximate height of the stack of pads. Now allow the stack to relax, undisturbed for the remainder of the relaxation time of 2 minutes. After 2 minutes have elapsed, measure the stack height with the UPT in the same manner as described for the FBW measurement, where the compression plate moves from the pre-set start position toward the stack at a rate of 12 mm/min until a force of 1 N force is applied to the stack and then the plate immediately moves back to the pre-set start position. The height of the compression plate at which the 1 N force was reached is recorded as Recovered Stack Height at 2 minutes to the nearest 0.1 mm. Now allow the stack of pads to sit on the base plate of the UPT, undisturbed, for a relaxation time of 4 hours. During this time, measure the mass of the empty package and record as empty bag mass (EBM) to the nearest 0.01 g. After 4 hours have elapsed, measure the stack height with the UPT as previously described. The height of the compression plate at which the 1 N force was reached is recorded as Recovered Stack Height at 4 hours to the nearest 0.1 mm. Now remove one of the pads from the stack, unfold it and record the number of folds as Folds Per Pad (FPP) to the nearest 1 fold. For example, a tri-folded pad will have a FPP equal to 3.

The following In-Bag calculations are performed to determine the pad density and pad caliper. Calculate the volume of the full bag test package as FBW×FBH×FBD and record as full bag volume (FBV) to the nearest 0.1 mm³. Now calculate the volume of an individual stack of pads inside the test package by dividing the full bag volume (FBV) by the stacks per bag (SPB) and record as In-Bag Stack Volume to the nearest 0.1 mm³. Calculate the In-Bag Stack Mass by subtracting the empty bag mass (EBM) from the filled bag mass (FBM) and then divide by stacks per bag (SPB) and record to the nearest 0.01 g. Now divide the In-Bag Stack Mass by the # of Pads Per Stack, then divide by the In-Bag Stack Volume divided by the # of Pads Per Stack, then multiply by 1000 and record as Average In-Bag Pad Density to the nearest 0.01 g/cm³. Calculate the In-Bag Folded Pad Caliper by dividing the In-Bag Stack Height (FBW) value by the # of Pads Per Stack in Bag value and record to the nearest 0.1 mm. Now divide the In-Bag Folded Pad Caliper value by the folds per pad (FPP) and record as Average In-Bag Pad Caliper to the nearest 0.1 mm.

The following Out-of-Bag calculations are performed to determine the pad caliper and density after the specified relaxation times. Calculate the Out-of-Bag Folded Pad Caliper at 2 minutes by dividing the Recovered Stack Height at 2 minutes value by the # of Pads Per Stack in Bag value and record to the nearest 0.1 mm. Now divide the Out-of-Bag Folded Pad Caliper at 2 minutes value by the folds per pad (FPP) and record as Average Out-of-Bag Pad Caliper at 2 minutes to the nearest 0.1 mm. Calculate the Out-of-Bag Stack Volume at 2 minutes by multiplying together the Recovered Stack Height at 2 minutes, the filled bag height (FBH) and the filled bag depth (FBD), then dividing by the stacks per bag (SPB) and record to the nearest 0.1 mm³. Now calculate the pad density at 2 minutes by dividing the In-Bag Stack Mass by the Out-of-Bag Stack Volume at 2 minutes, then multiply by 1000 and record as Average Out-of-Bag Pad Density at 2 minutes to the nearest 0.01 g/cm³. In like fashion, calculate the Average Out-of-Bag Pad Caliper at 4 hours and the Average Out-of-Bag Pad Density at 4 hours, recording to the nearest 0.1 mm and the nearest 0.01 g/cm³, respectively.

Calculate the percent caliper recovery after 2 minutes and 4 hours as follows and record each value to the nearest 1 percent.

$$\% Caliper Recovery at 2 minutes = \frac{\begin{matrix}Avg Out - of - Bag Pad Caliper at \\ 2 minutes - Avg In Bag Pad Caliper\end{matrix}}{Avg In Bag Pad Caliper} * 100 \\ \% Caliper Recovery at 4 hours = \frac{\begin{matrix}Avg Out - of - Bag Pad Caliper at \\ 4 hours - Avg In Bag Pad Caliper\end{matrix}}{Avg In Bag Pad Caliper} * 100$$

In like fashion, repeat the entire procedure until a total of three replicate full bag test packages are measured. Calculate the arithmetic mean of all three replicates and report In-Bag Stack Height to the nearest 0.1 mm, Average In-Bag Pad Caliper to the nearest 0.1 mm, Average In-Bag Pad Density to the nearest 0.01 g/cm³, Average Out-of-Bag Pad Caliper at 2 minutes to the nearest 0.1 mm, Average Out-of-Bag Pad Density at 2 minutes to the nearest 0.01 g/cm³, Average Out-of-Bag Pad Caliper at 4 hours to the nearest 0.1 mm, Average Out-of-Bag Pad Density to the nearest 0.01 g/cm³, % Caliper Recovery at 2 minutes to the nearest 1 percent and % Caliper Recovery at 4 hours to the nearest 1 percent.

### EXAMPLES/DATA

The following data and examples, including comparative examples, are provided to help illustrate the upper and lower nonwoven layers and/or absorbent articles described herein. The exemplified structures are given solely for the purpose of illustration and are not to be construed as limitations of the present disclosure, as many variations thereof are possible without departing from the spirit and scope of the invention.

### Nonwoven Layer Material Test

A series of measurements are performed on nonwoven materials to assess the ability of the material to function as an upper nonwoven layer and/or a lower nonwoven layer in the absorbent core structure described herein. Samples A-G are examples in accordance with the present disclosure. Comparative Sample H is a comparative example. Samples A-G and Comparative Sample H are described in Table 1 below.

Samples A-G and Comparative Sample H are evaluated according to the CD Cyclic Elongation to 3% Strain Method, the Strain to Break Method, Wet and Dry CD Ultra Sensitive 3 Point Bending Method, and the Nonwoven Thickness-Pressure Method. The results are shown in Table 2.

TABLE 1
Nonwoven Material Description

| Sample | Nonwoven Material | Fiber Composition |
|---|---|---|
| A | 40 gsm Carded Resilient Nonwoven¹ | BiCo (PE/PET) - 60% 2 DTex/ |
| | | 40% 4 DTex Blend |
| B | 55 gsm Resilient Spunlace 1² | 30% 10 DTex HS-PET; |
| | | 20% 1.3 DTex Rayon; |
| | | 50% 2.2 DTex BiCo (PE/PET) |
| C | 50 gsm Resilient Spunlace 6³ | 20% 1.3 DTex Rayon; |
| | | 20% 3.3 DTex tri-lobal Rayon; |
| | | 60% 5.8 DTex PE/PET |
| D | 24 gsm Carded Nonwoven⁴ | 100% 2 DTex BiCo (PE/PET) |
| E | 55 gsm Resilient Spunlace 5⁵ | 40% 1.7 DTex/38 mm Rayon; |
| | | 40% 2.2 DTex PET; |
| | | 20% 10 DTex HS PET |
| F | 18 gsm Spunbond Nonwoven⁶ | 100% 2.0 DTex BiCo (PE/PP) |
| G | 25 gsm Spunbond Nonwoven⁷ | 100% 2.0 DTex BiCo (PE/PP) |
| Comparative | 17 gsm Tissue⁸ | 100% Cellulose |
| Sample H | | |

¹Available as ATB Z87G-40 from Xiamen Yanjan New Material Co. (China)
²Available as Sawasoft ® 53FC041001 from Sandler GmbH (Germany)
³Available as Sawasoft ® 553FC041005 (option 82) from Sandler GmbH (Germany)
Available as Aura 20 from Xiamen Yanjan New Material Co. (China)
⁵Available as S25000541R01 from Jacob Holms Industries (Germany)
⁶Available as PFNZN 18G BICO8020 PHI 6 from PFN nonwovens Czech S.R.O (Czech Republic)
⁷Available as PEGZN25 BICO7030 Phobic from PFN nonwovens Czech S.R.O (Czech Republic)
⁸Available as 3028 from DunnPaper (USA)

TABLE 2

| | | | Dry | Dry | | | |
|---|---|---|---|---|---|---|---|
| | Permanent | % Strain | Bending | Recovery | Density at | Thickness | Thickness |
| | Strain | to Break | Energy | Energy | 7 g/cm² | at 7 g/cm² | at 70 g/cm² |
| Sample | mm/mm | % | N*mm | N*mm | g/cm³ | mm | mm |
| A | 0.006 | >10% | 0.219 | 0.092 | 0.03 | 1.21 | 0.26 |
| B | 0.006 | >10% | 1.015 | 0.291 | 0.06 | 0.99 | 0.44 |
| C | 0.005 | >10% | 0.595 | 0.201 | 0.07 | 0.76 | 0.62 |
| D | 0.016 | >10% | 0.176 | 0.036 | 0.05 | 0.45 | 0.19 |
| E | 0.013 | >10% | 0.059 | 0.032 | 0.07 | 0.80 | 0.40 |
| F | 0.010 | >10% | 0.022 | 0.005 | 0.09 | 0.21 | 0.17 |
| G | 0.009 | >10% | 0.062 | 0.019 | NA | NA | NA |
| Comp. H | 0.014 | <5% | 0.073 | 0.031 | 0.12 | 0.14 | 0.11 |

It is believed that nonwoven materials suitable for upper and/or lower nonwoven layers can strain (elongate) with a balanced stretch and substantially recover to their original state, thus helping to enable the absorbent core structure and/or absorbent article to recover from deformation during bodily motions. Particularly suitable nonwoven materials of the upper nonwoven layer can provide fluid handling performance that can effectively transport fluid deep into the inner core layer to help provide a close and comfortable fit to the body that feels dry. To achieve this, suitable nonwoven materials of the upper nonwoven layer exhibit a relatively low density (e.g., from 0.03 to 0.07 g/cm3 under a pressure of 7 g/cm2) in order to allow fluid to efficiently drain from the upper nonwoven layer into the inner core layer below. In addition, suitable nonwoven materials of the upper nonwoven layer can maintain a relatively lofty thickness even under high bodily compressive forces (i.e., a pressure of 70 g/cm2) so that fluid residing within the inner core layer is not expelled back out of the absorbent core structure which can create a wet feeling on the body.

It was found that Samples A-C and E are suitable materials for upper and/or lower nonwoven layers. In particular, Samples A-C and E had a Permanent Strain of 0.013 mm/mm or less, demonstrating that the materials can elongate and recover, and had a Strain to Break of greater than 10% before tearing. Samples A-C and E also required less energy to bend (demonstrated by a Dry Bending Energy of less than 1.6 N*mm) while recovering from bending with a Dry Recovery Energy of greater than 0.03 N*mm. Samples A-C and E exhibited a relatively low density of from 0.03 to 0.07 g/cm³ under a pressure of 7 g/cm² and a Thickness at 7 g/cm² of pressure of from 0.80 to 1.21 mm, demonstrating that these materials are both lofty and have a more open fiber network structure that can help with efficient fluid handling performance.

It was found that Sample D had a Permanent Strain of 0.016 mm/mm, demonstrating that the material will likely elongate strongly during manufacture and/or in-use without recovering to its initial state. At the same time, Sample D was found to be highly compressible under bodily pressures, as demonstrated by a Thickness at 70 g/cm2 of 0.19 mm, illustrating that the material will become more dense under bodily compression and likely will not be sufficiently drained of fluid by the inner core layer. Samples F and G exhibited a Dry Recovery Energy of less than 0.03 N*mm, demonstrating that the materials may not recover from deformation, making the material insufficient to function as an upper nonwoven layer. However, when combined with an upper nonwoven layer described herein, Samples D, F, and G may be suitable materials for a lower nonwoven layer.

Comparative Sample H exhibited a Strain to Break of less than 5% and a Thickness at 70 g/cm2 of less than 0.2 mm. In addition, it was found that Comparative Sample H tears when wet. As such, Comparative Sample H is insufficient to function as an upper or lower nonwoven layer.

Table 3 is provided for the convenience of the reader. Table 3 includes a non-exhaustive list of properties as well as a non-exhaustive list of corresponding values for each of the properties that particularly suitable upper nonwoven layers of the present disclosure may exhibit.

TABLE 3
Upper Nonwoven Layer

| Property | Value |
|---|---|
| Permanent Strain | 0.005 to 0.013 mm/mm |
| % Strain to Break | Greater than 10% |
| Dry Recovery Energy | Greater than 0.03 N*m |
| Density at 7 g/cm² | 0.03 to 0.07 g/cm³ |
| Thickness at 7 g/cm² | 0.3 mm to 1.3 mm |
| Thickness at 70 g/cm² | 0.2 mm to 0.7 mm |

### In-Bag Compression Test

Absorbent products are tested to assess the impact of in-bag compression on absorbent article caliper and density. The following examples provide a comparison between commercially available feminine hygiene products to feminine hygiene products according to the present disclosure.

### Examples 1-5

Disposable absorbent articles in the form of a feminine hygiene pad (280 mm length) as described herein are prepared having the following components:
Topsheet—the topsheet is 24 gsm carded nonwoven comprising 100% 4 Denier BiCo (PE/PET), available from Xiamen Yanjan New Material Co.
Upper nonwoven layer—the upper nonwoven layer is a 40 gsm carded resilient nonwoven comprising BiCo (PE/PET) in a 60% 2 DTex/40% 4 DTex blend, available as ATB Z87G-40 from Xiamen Yanjan New Material Co. (China).
Inner core layer—the inner core layer is a homogeneous mixture of 180 gsm cellulose pulp (100% SuperSoft® untreated fluff pulp, available from International Paper Company (Memphis, TN)) and 70 gsm superabsorbent particles (available as Aqualic CA L-805 from Nippon Shokubai (Japan)).
Lower nonwoven layer—the lower nonwoven layer is an 18 gsm spunbond nonwoven comprising 100% 2.0 DTex polypropylene, available as PFNZN 18G 100% PP PHI 6 from PFN nonwovens Czech S.R.O (Czech Republic).
Backsheet—the backsheet is a 12 gsm polyolefin resin based blow film of metallocene-LLDPE, LDPE and HDPE composition (3 layers), available as Theo 12 gsm film from RKW Group (Germany).

First, the topsheet is coated with 5 gsm adhesive (D3151 NG available from HB Fuller). The upper nonwoven layer is then deposited onto the topsheet. In parallel, the inner core layer is produced in an airlaying process. Streams of cellulose fiber and AGM are carried on a fast-moving airstream and deposited into a three dimensionally shaped pocket on a rotating forming drum with a vacuum below to draw the cellulose and AGM into the pocket in a laydown station. The homogeneously mixed cellulose and AGM mass is held on the forming drum under vacuum until it is directly deposited onto the upper nonwoven layer that has been pre-coated with 5 gsm of adhesive (D3151 NG, HB Fuller) and then sealed with the second remaining nonwoven, precoated with 5 gsm of an adhesive (D3151 NG, HB Fuller) to create the absorbent core. The width of the upper and lower nonwoven webs is wider than the maximum width of the shaped cellulose and AGM inner core layer so as to enable an effective perimeter seal where the two nonwoven layers connect, at least on the left and right most sides of the absorbent core structure. Flex bond channel regions are applied with the pattern shown in FIG. 11B using a heated embossing unit on a manufacturing line. The backsheet is then bonded to the outward facing surface of the lower nonwoven layer with 5 gsm adhesive (D3151 NG, HB Fuller).

Panty fastening adhesive is applied to the garment-facing surface of the pad and then covered with siliconized Kraft paper. The pad is tri-folded and then wrapped and sealed in an un-siliconized 14 gsm polyolefin film pouch. A stack of 10 folded pads are placed in poly bag packages. The packages are compressed to various in-bag compression levels and sealed.

The packages were evaluated according to the In Bag Compression Recovery Method and the results are shown in Tables 4a and 4b.

TABLE 4a
In Bag

| | | In-Bag | | Avg In-Bag | Avg In-Bag |
|---|---|---|---|---|---|
| | | Stack | In-Bag | Pad | Pad |
| | In-Bag | Height | Stack Mass | Density | Caliper |
| EX | Compression | mm | g | g/cm3 | mm |
| 1 | 0% | 118.4 | 86.14 | 0.10 | 3.9 |
| 2 | 5% | 112.5 | 85.19 | 0.10 | 3.8 |
| 3 | 15% | 98.5 | 86.54 | 0.11 | 3.3 |
| 4 | 20% | 90.6 | 83.00 | 0.11 | 3.0 |
| 5 | 25% | 86.9 | 86.33 | 0.12 | 2.9 |

TABLE 4b
Recovery

| | After 2 minutes | | | | After 4 hours | | | |
|---|---|---|---|---|---|---|---|---|
| | | Avg | Avg | | | Avg | Avg | |
| | Recovered | Out-of- | Out-of- | % | Recovered | Out-of- | Out-of- | % |
| | Stack | Bag Pad | Bag Pad | Caliper | Stack | Bag Pad | Bag Pad | Caliper |
| | Height | Density | Caliper | Recovery | Height | Density | Caliper | Recovery |
| EX | mm | g/cm3 | mm | % | mm | g/cm3 | mm | % |
| 1 | 118.4 | 0.10 | 3.9 | 0% | 118.4 | 0.10 | 3.9 | 0% |
| 2 | 117.2 | 0.09 | 3.9 | 4% | 117.4 | 0.09 | 3.9 | 4% |
| 3 | 111.7 | 0.10 | 3.7 | 13% | 112.8 | 0.10 | 3.8 | 15% |
| 4 | 107.4 | 0.10 | 3.6 | 19% | 108.4 | 0.09 | 3.6 | 20% |
| 5 | 107.0 | 0.10 | 3.6 | 23% | 109.5 | 0.10 | 3.7 | 26% |

It was surprisingly found that the feminine hygiene pads described herein can be compressed and held in packages without negatively impacting caliper or density. After 2 minutes from removal from the package, the feminine hygiene pads of Examples 2-5 exhibited a % Caliper Recovery of from 4% to 23%. After 4 hours from removal from the package, the feminine hygiene pads of Examples 3-5 continued to recover caliper, exhibiting a % Caliper Recovery of from 15% to 26%. The feminine hygiene pads of Examples 2-5 exhibited a density both in-bag and out-of-bag of less than 0.16 g/cm³. As such, Examples 2-5 demonstrate the absorbent articles described herein can be compressed and held in a smaller package that requires less packaging material, reduced shipping volume, and lower transportation and storage costs while nevertheless substantially recovering to their pre-packaged caliper and density.

Example 6 is a commercially available Always® Ultra Thin feminine hygiene product sold by The Procter & Gamble Company in Western Europe (Size 1 Normal; bag count 13; Lot #D DE 3 291 0314 20 08:33 P:181023).

Example 7 is a commercially available Bodyform® feminine hygiene product sold by Essity in Western Europe (Size 1; bag count 12; Lot #160923 GH 16 20:42).

Example 8 is a commercially available Always® Maxi feminine hygiene product sold by The Procter & Gamble Company in Western Europe (Size 2 Long; bag count 12; Lot #HU 3048208001 11 18:45 PROD: 17/02/23 7657).

Example 9 is a commercially available Sofy® Antibacteria Slim feminine hygiene product sold by Unicharm in India (Size XL 290 mm; bag count 14; Lot #SEP22 01/09/22 B21F3U).

Example 10 is a commercially available Sofy® Naked Feel feminine hygiene product sold by Unicharm in China (25 cm pad length; bag count 13; Lot #20200120D2082).

Example 11 is a commercially available Always® Ultra Thin feminine hygiene product sold by The Procter & Gamble Company in North America (size 3 Extra Long Super; bag count 32 (2×16); Lot #230047860063 3010032).

Examples 6-11 were evaluated according to the In Bag Compression Recovery Method and the results are shown in Tables 5a and 5b.

TABLE 5a
In Bag

| | In-Bag | | Avg In-Bag | Avg In-Bag |
|---|---|---|---|---|
| | Stack | In-Bag | Pad | Pad |
| | Height | Stack Mass | Density | Caliper |
| EX | mm | g | g/cm3 | mm |
| 6 | 91.2 | 55.23 | 0.08 | 2.4 |
| 7 | 77.8 | 65.98 | 0.10 | 2.2 |
| 8 | 159.8 | 111.59 | 0.08 | 4.5 |
| 9 | 148.8 | 128.98 | 0.09 | 3.6 |
| 10 | 100 | 97.02 | 0.14 | 2.6 |
| 11 | 120 | 91.04 | 0.10 | 2.9 |

TABLE 5b
Recovered

| | After 2 minutes | | | | After 4 hours | | | |
|---|---|---|---|---|---|---|---|---|
| | | Avg | Avg | | | Avg | Avg | |
| | Recovered | Out-of- | Out-of- | % | Recovered | Out-of- | Out-of- | % |
| | Stack | Bag Pad | Bag Pad | Caliper | Stack | Bag Pad | Bag Pad | Caliper |
| | Height | Density | Caliper | Recovery | Height | Density | Caliper | Recovery |
| EX | mm | g/cm³ | mm | % | mm | g/cm³ | mm | % |
| 6 | 89.5 | 0.08 | 2.3 | −4 | 90.5 | 0.08 | 2.4 | 0 |
| 7 | 78.2 | 0.10 | 2.2 | 0 | 78.9 | 0.10 | 2.2 | 0 |
| 8 | 158.3 | 0.08 | 4.4 | −2 | 159.2 | 0.08 | 4.5 | 0 |
| 9 | 145.9 | 0.09 | 3.5 | −3 | 145.8 | 0.09 | 3.5 | −3 |
| 10 | 105 | 0.13 | 2.7 | 3 | 86.4 | 0.15 | 2.4 | −8 |
| 11 | 115 | 0.12 | 2.7 | −6 | 100.8 | 0.12 | 2.4 | −17 |

Examples 6-11, which are commercially available feminine hygiene products, did not recover caliper and or density after 4 hours from removal from the package.

Absorbent products are tested to assess the impact of in-bag compression on the ability of the pad to compress and to recover to their original state and on fluid handling performance.

### Examples 12-13

Disposable absorbent articles in the form of a feminine hygiene pad (280 mm length) as described herein are prepared having the following components:
Topsheet—the topsheet is 22.4 gsm polyethylene formed film, available as DS02-172 from Xiamen Yanjan New Material Co. (India).
Upper nonwoven layer—the upper nonwoven layer is a 40 gsm carded resilient nonwoven comprising BiCo (PE/PET) in a 60% 2 DTex/40% 4 DTex blend, available as ATB Z87G-40 from Xiamen Yanjan New Material Co. (China).
Inner core layer—the inner core layer is a homogeneous mixture of 150 gsm cellulose pulp (100% SuperSoft® untreated fluff pulp, available from International Paper Company (Memphis, TN)) and 60 gsm superabsorbent particles (available as Aqualic CA L-805 from Nippon Shokubai (Japan)).
Lower nonwoven layer—the lower nonwoven layer is an 18 gsm spunbond nonwoven comprising 100% 2.0 DTex polypropylene, available as PFNZN 18G 100% Polypropylene PHI 6 from PFN nonwovens Czech S.R.O (Czech Republic).
Backsheet—the backsheet is 12 gsm polyolefin resin-based blow film of metallocene-LLDPE, LDPE and HDPE composition (3 layers), available as Theo 12 gsm film from RKW Group, Germany.

Examples 12 and 13 are produced as described above for Examples 1-5. Examples 12 and 13 were evaluated according to the Wet & Dry CD & MD 3 Point Bend Method and Wet and Dry Bunched Compression Method, with the results shown in Table 6, and the Acquisition Time and Rewet Method, with the results shown in Table 7.

TABLE 6

| | | Wet & Dry CD & MD 3 Point | | | | Wet and Dry Bunched | |
|---|---|---|---|---|---|---|---|
| | | Bend Method | | | | Compression Method | |
| | | | | | CD Dry | 5^{th} Cycle | 5^{th} Cycle |
| | In-Bag | Dry | Dry | CD Dry | Bending | Wet Energy | Wet % |
| | Compression | Caliper | Density | Modulus | Stiffness | of Recovery | Recovery |
| Ex | (%) | (mm) | (g/cm³) | (N/mm²) | (N*mm²) | (N*mm) | (%) |
| 11 | 0 | 3.3 | 0.107 | 0.052 | 7.9 | 1.8 | 32 |
| 12 | 26 | 3.2 | 0.119 | 0.114 | 15.5 | 2.2 | 30 |

TABLE 7

| | | Acquisition Time and Rewet Method | | | |
|---|---|---|---|---|---|
| | | Total Gush | Surface | Interfacial | Total |
| | In-Bag | Absorbency | Free Fluid | Free Fluid | IFF + |
| | Compression | Time | (SFF) | (IFF) | SFF |
| Ex | (%) | (seconds) | (mg) | (mg) | (mg) |
| 11 | 0 | 5.0 | 10.2 | 22.3 | 32.5 |
| 12 | 26 | 5.3 | 10.6 | 20.2 | 30.7 |

It was found that prolonged compression (Example 12) did not have a negative impact on the ability of the article to bend and/or compress and recover to the original shape. It was also found that even after being held in a package at a 26% in-bag compression level, Example 12 can efficiently manage fluid at levels comparable to Example 11 which was not compressed.

### Combinations/Examples

Paragraph A. A feminine hygiene product comprising:
a package comprising an interior space and an exterior surface;
   a plurality of disposable feminine hygiene pads disposed within the interior space of the package, each of the disposable feminine hygiene pads comprising an absorbent core structure, wherein the absorbent core structure comprises an upper nonwoven layer, a lower nonwoven layer, and an inner core layer comprising cellulosic fibers, wherein at least a portion of the inner core layer is disposed between the upper nonwoven layer and the lower nonwoven layer,
   wherein the disposable feminine hygiene pads exhibit an Average In-Bag Pad Density of about 0.20 g/cm³ or less, and a % Caliper Recovery at 2 minutes of at least 4%, preferably of at least 10%, as measured according to the In Bag Compression Recovery Method.
      Paragraph B. The feminine hygiene product of Paragraph A, wherein the disposable feminine hygiene pads exhibit an Average In-Bag Pad Density of from about 0.07 g/cm³ to about 0.17 g/cm³, as measured according to the In Bag Compression Recovery Method.
      Paragraph C. The feminine hygiene product of Paragraph A or B, wherein the disposable feminine hygiene pads exhibit a % Caliper Recovery at 2 minutes of from about 4% to about 35%, as measured according to the In Bag Compression Recovery Method.
      Paragraph D. The feminine hygiene product of any of Paragraphs A-C, wherein the disposable feminine hygiene pads exhibit a % Caliper Recovery at 4 hours of from about 4% to about 35%, as measured according to the In Bag Compression Recovery Method.
      Paragraph E. The feminine hygiene product of any of Paragraphs A-D, wherein the inner core layer comprises from about 50% to about 85% cellulosic fibers, by weight of the inner core layer.
      Paragraph F. The feminine hygiene product of any of Paragraphs A-E, wherein the inner core layer further comprises from about 15% to about 50% superabsorbent particle, by weight of the inner core layer.
      Paragraph G. The feminine hygiene product of any of Paragraphs A-F, wherein the upper nonwoven layer has a basis weight of from about 30 gsm to about 85 gsm.
      Paragraph H. The feminine hygiene product of any of Paragraphs A-G, wherein the lower nonwoven layer has a basis weight of from about 7 gsm to about 40 gsm.
      Paragraph I. The feminine hygiene product of any of Paragraphs A-H, wherein the feminine hygiene pads exhibit an Average Out-of-Bag Pad Density at 2 minutes of from about 0.06 g/cm³ to about 0.16 g/cm³, as measured according to the In Bag Compression Recovery Method.
      Paragraph J. The feminine hygiene product of any of Paragraphs A-I, wherein the package comprises a package material selected from the group consisting of a polymer film, a paper material, carton board, and combinations thereof.
      Paragraph K. The feminine hygiene product of any of Paragraphs A-J, wherein the feminine hygiene pads exhibit a CD Dry Bending Stiffness between about 7.0 N*mm2 to about 30 N*mm² as measured according to the Wet and Dry CD and MD 3 Point Bend Method.
      Paragraph L. The feminine hygiene product of any of Paragraphs A-K, wherein the feminine hygiene pads exhibit an Average In-Bag Pad Caliper of from about 2.0 mm to about 5.5 mm, as measured according to the In Bag Compression Recovery Method.
      Paragraph M. The feminine hygiene product of any of Paragraphs A-L, wherein at least one of the upper nonwoven layer and the lower nonwoven layer comprise a polymer selected from the group consisting of a recycled polymer resin, a biodegradable polymer, biopolymers, and combinations thereof.
      Paragraph N. The feminine hygiene product of any of Paragraphs A-M, wherein the polymer fibers of the upper nonwoven layer comprise from about 60 to about 100% synthetic fibers, and from 0 to 40% regenerated cellulosic fibers comprising rayon.
      Paragraph O. The feminine hygiene product of any of Paragraphs A-N, each of the disposable feminine hygiene pads further comprise a topsheet and a backsheet; wherein the absorbent core structure is disposed between the topsheet and the backsheet.
      Paragraph P. A process for packaging a plurality of disposable feminine hygiene pads, the process comprising:
providing a plurality of disposable feminine hygiene pads, each of the disposable feminine hygiene pads comprising an absorbent core structure, the absorbent core structure comprising an upper nonwoven layer comprising polymer fibers and having a basis weight of from about 30 gsm to about 85 gsm, a lower nonwoven layer comprising polymer fibers and having a basis weight of from about 7 gsm to about 40 gsm, and an inner core layer comprising cellulosic fibers and superabsorbent particles, wherein at least a portion of the inner core layer is disposed between the upper nonwoven layer and the lower nonwoven layer;
folding each disposable feminine hygiene pad of the plurality of disposable feminine hygiene pads to form a plurality of folded disposable feminine hygiene pads;
arranging the plurality of folded disposable feminine hygiene pads to form a stack of folded disposable feminine hygiene pads;
compressing the stack of folded disposable feminine hygiene pads along a compression axis to form a compressed stack of folded disposable feminine hygiene pads;
placing the compressed stack of folded disposable feminine hygiene pads in an interior space of a package, wherein the compressed stack of folded disposable feminine hygiene pads is placed in the interior space of the package such that the compression axis of the stack of folded disposable feminine hygiene pads is oriented substantially along the width dimension of the package; and
closing the package such that the folded disposable feminine hygiene pads exhibit an Average In-Bag Folded Caliper from about 7.0 to about 15.0 mm, and such that upon removal from the package, the disposable feminine hygiene pads exhibit a % Caliper Recovery at 2 minutes of at least 4%, as measured according to the In Bag-Compression Recovery Method.
   Paragraph Q. The feminine hygiene product of Paragraph P, each of the disposable feminine hygiene pads further comprise a topsheet and a backsheet; wherein the absorbent core structure is disposed between the topsheet and the backsheet.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A feminine hygiene product comprising:
a package comprising an interior space and an exterior surface;
a plurality of disposable feminine hygiene pads disposed within the interior space of the package, each of the disposable feminine hygiene pads comprising a topsheet, a backsheet, and an absorbent core structure disposed between the topsheet and the backsheet, wherein the absorbent core structure comprises an upper nonwoven layer, a lower nonwoven layer, and an inner core layer comprising cellulosic fibers, wherein at least a portion of the inner core layer is disposed between the upper nonwoven layer and the lower nonwoven layer,
wherein the disposable feminine hygiene pads exhibit an Average In-Bag Pad Density of about 0.20 g/cm³ or less, and a % Caliper Recovery at 2 minutes of at least 4%, as measured according to the In Bag Compression Recovery Method.

2. The feminine hygiene product of claim 1, wherein the disposable feminine hygiene pads exhibit an Average In-Bag Pad Density of from about 0.07 g/cm³ to about 0.17 g/cm³, as measured according to the In Bag Compression Recovery Method.

3. The feminine hygiene product of claim 1, wherein the disposable feminine hygiene pads exhibit a % Caliper Recovery at 2 minutes of at least 10%.

4. The feminine hygiene product of claim 1, wherein the inner core layer comprises from about 50% to about 85% cellulosic fibers, by weight of the inner core layer.

5. The feminine hygiene product of claim 1, wherein the inner core layer further comprises from about 15% to about 50% superabsorbent particle, by weight of the inner core layer.

6. The feminine hygiene product of claim 1, wherein the upper nonwoven layer has a basis weight of from about 30 gsm to about 85 gsm.

7. The feminine hygiene product of claim 6, wherein the lower nonwoven layer has a basis weight of from about 7 gsm to about 40 gsm.

8. A feminine hygiene product comprising:
a package comprising an interior space and an exterior surface;
a plurality of disposable feminine hygiene pads disposed within the interior space of the package, each of the disposable feminine hygiene pads comprising a topsheet, a backsheet, and an absorbent core structure disposed between the topsheet and the backsheet, the absorbent core structure comprising an upper nonwoven layer comprising polymer fibers and having a basis weight of from about 30 gsm to about 85 gsm, a lower nonwoven layer comprising polymer fibers and having a basis weight of from about 7 gsm to about 40 gsm, and an inner core layer comprising a mixture of cellulosic fibers and superabsorbent particles, wherein at least a portion of the inner core layer is disposed between the upper nonwoven layer and the lower nonwoven layer;
wherein the feminine hygiene pads exhibit an Average In-Bag Pad Density of 0.20 g/cm³ or less, measured according to the In Bag Compression Recovery Method.

9. The feminine hygiene product of claim 8, wherein the disposable feminine hygiene pads exhibit a % Caliper Recovery at 4 hours of from about 4% to about 35%, as measured according to the In Bag Compression Recovery Method.

10. The feminine hygiene product of claim 8, wherein the package comprises a package material selected from the group consisting of a polymer film, a paper material, carton board, and combinations thereof.

11. The feminine hygiene product of claim 8, wherein the inner core layer comprises from about 125 gsm to about 350 gsm cellulosic fibers.

12. The feminine hygiene product of claim 11, wherein the inner core layer comprises from about 20 gsm to about 125 gsm superabsorbent particles.

13. The feminine hygiene product of claim 8, wherein the feminine hygiene pads exhibit a CD Dry Bending Stiffness between about 7.0 N*mm2 to about 30 N*mm2 as measured according to the Wet and Dry CD and MD 3 Point Bend Method.

14. The feminine hygiene product of claim 8, wherein the absorbent articles exhibit a 5th Cycle Wet % Recovery of between about 29% and about 40% as measured according to the Wet and Dry Bunched Compression Method.

15. A feminine hygiene product of comprising:
a package comprising an interior space and an exterior surface;
a plurality of disposable feminine hygiene pads disposed within the interior space of the package, each of the disposable feminine hygiene pads comprising a topsheet, a backsheet, and an absorbent core structure disposed between the topsheet and the backsheet, the absorbent core structure comprising an upper nonwoven layer comprising polymer fibers and having a basis weight of from about 30 gsm to about 85 gsm, a lower nonwoven layer comprising polymer fibers, and an inner core layer comprising from about 50% to about 85% cellulosic fibers, by weight of the inner core layer, and from about 15% to about 50% superabsorbent particle, by weight of the inner core layer, wherein at least a portion of the inner core layer is disposed between the upper nonwoven layer and the lower nonwoven layer;
wherein the feminine hygiene pads exhibit a % Caliper Recovery at 2 minutes of from about 4% to about 35%, as measured according to the In Bag Compression Recovery Method.

16. The feminine hygiene product of claim 15, wherein the feminine hygiene pads exhibit an Average In-Bag Pad Caliper of from about 2.0 mm to about 5.5 mm, as measured according to the In Bag Compression Recovery Method.

17. The feminine hygiene product of claim 15, wherein the feminine hygiene pads exhibit an Average Out-of-Bag Pad Caliper at 2 minutes of from about 2.0 to about 6.0 mm, as measured according to the In Bag Compression Recovery Method.

18. The feminine hygiene product of claim 15, wherein the feminine hygiene pads exhibit an Average In-Bag Pad Density of from about 0.07 g/cm³ to about 0.20 g/cm³ as measured according to the In Bag Compression Recovery Method.

19. The feminine hygiene product of claim 15, wherein the feminine hygiene pads exhibit an Average Out-of-Bag Pad Density of from about 0.06 g/cm³ to about 0.16 g/cm³ at 2 minutes, as measured according to the In Bag Compression Recovery Method.

20. A process for packaging a plurality of disposable feminine hygiene pads, the process comprising:
providing a plurality of disposable feminine hygiene pads, each of the disposable feminine hygiene pads comprising a topsheet, a backsheet, and an absorbent core structure disposed between the topsheet and the backsheet, the absorbent core structure comprising an upper nonwoven layer comprising polymer fibers and having a basis weight of from about 30 gsm to about 85 gsm, a lower nonwoven layer comprising polymer fibers and having a basis weight of from about 7 gsm to about 40 gsm, and an inner core layer comprising cellulosic fibers and superabsorbent particles, wherein at least a portion of the inner core layer is disposed between the upper nonwoven layer and the lower nonwoven layer;
folding each disposable feminine hygiene pad of the plurality of disposable feminine hygiene pads to form a plurality of folded disposable feminine hygiene pads;
arranging the plurality of folded disposable feminine hygiene pads to form a stack of folded disposable feminine hygiene pads;
compressing the stack of folded disposable feminine hygiene pads along a compression axis to form a compressed stack of folded disposable feminine hygiene pads;
placing the compressed stack of folded disposable feminine hygiene pads in an interior space of a package, wherein the compressed stack of folded disposable feminine hygiene pads is placed in the interior space of the package such that the compression axis of the stack of folded disposable feminine hygiene pads is oriented substantially along the width dimension of the package; and
closing the package such that the folded disposable feminine hygiene pads exhibit an In-Bag Folded Caliper from about 7.0 to about 15.0 mm, and such that upon removal from the package, the disposable feminine hygiene pads exhibit a % Caliper Recovery at 2 minutes of at least 4%, as measured according to the In Bag Compression Recovery Method.
